(19) United States
(12) Patent Application Publication
(10) Pub. No.: US 2024/0383840 A1
(43) Pub. Date: Nov. 21, 2024

(54) AMINE-N-OXIDE COMPOUNDS

(71) Applicants: Karl Franzens Universität Graz, Graz (AT); Rijksuniversiteit Groningen, CP Groningen (NL)

(72) Inventors: Katalin BARTA WEISSERT, Graz (AT); Markus HOCHEGGER, Graz (AT); Fridirich BÁLINT, AG Groningen (NL)

(21) Appl. No.: 18/693,845

(22) PCT Filed: Sep. 21, 2022
(86) PCT No.: PCT/EP2022/076255

§ 371 Date: Mar. 20, 2024

(30) Foreign Application Priority Data
Sep. 21, 2021 (EP) 21198124.6

(51) Int. Cl. C07C 209/60; C07D 207/46; C07D 211/94

(52) U.S. Cl. CPC C07C 209/60; C07D 207/46; C07D 211/94

(57) ABSTRACT

Amine N-oxide compounds having formula (I) or (II), a preparation method, and a surfactant containing the compounds:

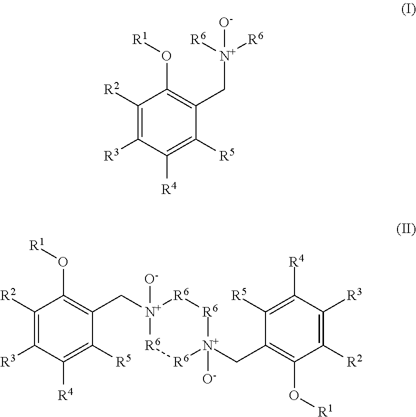

R¹ is a hydrocarbon residue with 4 to 26 carbon atoms and optionally at least one O or S; R², R³ and R⁵ are each hydrogen, R¹—O—, R⁸ and in formula (I) also optionally —CH₂—N⁺(O⁻)R⁶R⁶; R⁸ is a hydrocarbon radical with 1 to 26 carbon atoms and optionally at least one O or S; R⁴ is hydrogen or R⁸; and each R⁶ is a hydrocarbon radical with 1 to 6 carbon atoms and optionally at least one N, O or S. Optionally two radicals R⁶ on the same nitrogen atom are connected to form a five- or six-membered, nitrogen-containing ring, or optionally radical(s) R⁶ of a moiety —N⁺(O⁻)R⁶R⁶ may be linked to radical(s) R⁶ of another formula (I) molecule, forming a bridge having the structure

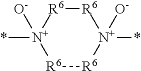

and a dimer of formula (II).

The present invention relates to a method for preparing novel amine N-oxide compounds, the compounds thus prepared and their use as surfactants.

### BACKGROUND OF THE INVENTION

Lignin is an abundant aromatic biopolymer, the structure of which is based primarily on three substituted phenols, so-called monolignols (p-coumaryl, coniferyl and sinapyl alcohol), which are characterized by a variety of different C—O and C—C bonds forming an amorphous 3-dimensional structure. Different methods have been developed to enable the catalytic degradation of lignin by means of depolymerization in order to obtain industrially usable monomolecular phenol and/or benzaldehyde derivatives. In addition to monomers, the products of these depolymerization processes may also contain phenolic di-, tri- and oligomers.

Only recently, various methods have been developed through which selective depolymerization of lignin has become possible, mainly involving reductive or oxidative reaction strategies. The former usually yield phenol, guaiacol or syringol derivatives with aliphatic radicals being typically one to three carbon atoms in length and bearing alcohol, aldehyde, ester and/or ketone functionalities. The latter, on the other hand, typically provide aromatic aldehydes such as vanillin or syringaldehyde or similarly functionalized guaiacol and syringol derivatives. The main products of such depolymerization processes include guaiacol and syringol or vanillin and syringaldehyde, respectively, each of which often have one or more alkyl and/or alkoxy substituents on the aromatic ring.

All of the lignin degradation products mentioned represent valuable biologically based resources from which a number of different products have been produced in recent years. According to the inventors' research, there are hardly any surfactants, although, also in this area, there is a need for products that can be synthesized based on nonedible, renewable raw materials.

In literature, in addition to numerous other amphiphilic compounds, which generally consist of hydrophobic hydrocarbons with one or more ionic hydrophilic groups, such as carboxylic acid, sulfonic acid or quaternary ammonium salts, also zwitterionic amine N-oxides are disclosed for use as surfactants. However, these are practically exclusively N-oxides of fatty amines, i.e. of tertiary alkylamines with 8 or more, e.g. at least 12, carbon atoms. Among them, however, amine N-oxides with aromatic radicals are virtually impossible to find.

The inventors are currently only aware of a handful of publications in which such an amine N-oxide is disclosed as a surfactant, including, for example, Goracci et al., ChemBioChem 6(1), 197-203 (2005), Cesareti et al., Phys. Chem. Chem. Phys. 17(26), 17214-17220 (2015), and Gabriele et al., Langmuir 34(38), 11510-11517 (2018). In all cases, however, the same amine N-oxide, namely 4- or p-dodecyloxybenzyl-dimethylamine N-oxide (abbrev.: “pDoAO”), is always described and examined:

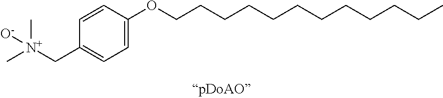 [SMILES: CCCCCCCCCCCCOc1ccc(C[N+](C)(C)[O-])cc1]

In Goracci et al. from 2005, the origin of this substance is mentioned to be a production process which includes the reaction of p-dodecyloxybenzyl bromide with dimethylamine and the subsequent oxidation of the amine with hydrogen peroxide according to the following scheme, with a combined yield for both stages of 88% being stated:

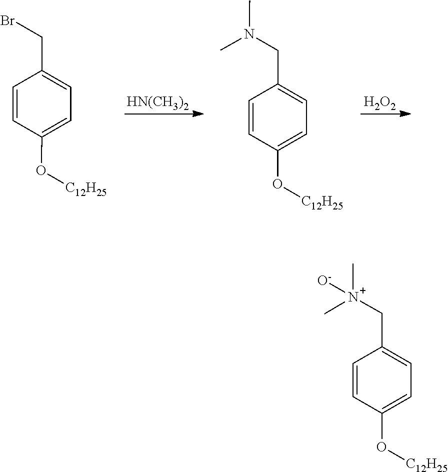

A few years later, Di Crescenzo et al., Eur. J. Org. Chem. 28, 5641-5648 (2011), also described the synthesis of p-dodecyloxybenzyl bromide starting from 4-hydroxybenzaldehyde, the OH group of which was first etherified with dodecyl bromide, whereafter the aldehyde group was reduced to OH using NaBH₄, and this was exchanged for bromine using PBr₃ as shown below (however, this bromide is not subsequently converted into the amine N-oxide pDoAO, but rather reacted with trimethylamine to form the quaternary ammonium salt p-dodecyloxybenzyltrimethylammonium bromide called pDoTABr):

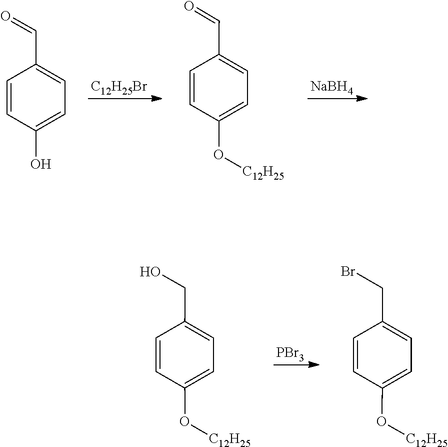

The combined yield for the first two of these three stages Di Crescenzo et al. state 85%, but for the final bromination the corresponding value is missing. In the case of an optimal yield of 100% for the bromination reaction, a total yield of about 75% results for the complete reaction sequence for the synthesis of pDoAO from 4-hydroxybenzaldehyde; however, with a more realistic assumption of around 95% yield for the bromination reaction, the overall yield is only around 70%. Although this is in principle not a bad value for a five-stage synthesis process, it is still not really satisfactory with regard to synthesis on an industrial scale.

In addition, 4-hydroxybenzaldehyde is not one of the usual lignin degradation products. Rather, as mentioned above, these mainly include phenols or benzaldehydes being multiply substituted with alkyl and/or alkoxy. 4-Hydroxybenzaldehyde is therefore not to be considered as an economic starting product and under no circumstances as a biologically based resource. The same applies to the reagents NaBH₄ and PBr₃ used in stoichiometric amounts in the above synthesis process for preparing pDoAO.

Against this background, the aim of the invention was the development of a novel synthesis process for the preparation of aromatic amine N-oxides suitable as surfactants by functionalizing products that arise in large quantities in the course of lignin degradation or similar compounds, and preferably in an environmentally friendly way.

### SUMMARY OF THE INVENTION

The present invention achieves this goal in a first aspect by providing a method for preparing an amine N-oxide compound according to the following formula (I) or (II):

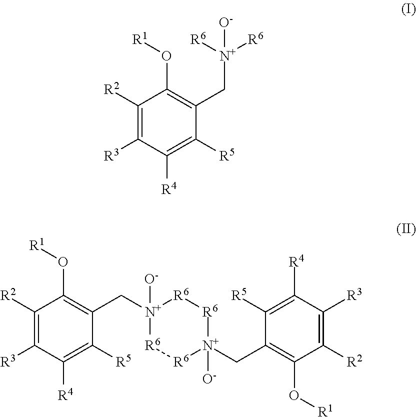

wherein
each R¹ is selected from linear, branched or cyclic hydrocarbon radicals having 4 to 26 carbon atoms, wherein optionally at least one carbon atom may be replaced by an oxygen or sulfur atom;
R², R³ and R⁵ are each independently selected from hydrogen, R¹—O—, R⁸, and in formula (I) also from —CH₂—N⁺(O⁻)R⁶R⁶, wherein R⁸ represents a linear, branched or cyclic hydrocarbon radical having 1 to 26 carbon atoms, wherein optionally at least one carbon atom may be replaced by an oxygen or sulfur atom;
R⁴ is selected from hydrogen and R⁸; and
each radical R⁶ is independently selected from saturated, linear or branched hydrocarbon radicals having 1 to 6 carbon atoms, wherein optionally at least one carbon atom may be replaced by a nitrogen, oxygen or sulfur atom;
wherein optionally two radicals R⁶ being bound to the same nitrogen atom may be connected to form a five- or six-membered, nitrogen-containing ring, or
wherein optionally one or both radical(s) R⁶ of an amine N-oxide moiety —N⁺(O⁻)R⁶R⁶ may be linked to one or both radical(s) R⁶ of such a moiety of another molecule of formula (I), thus forming a bridge having the structure

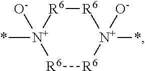

wherein the dashed line designates an optional bond between the two radicals R⁶ and the asterisks designate the connections of the bridge to the two aromatic rings, thereby forming a dimer according to formula (II);
wherein the inventive method comprising the following steps:
1) reacting a phenol derivative according to the following formula (III):

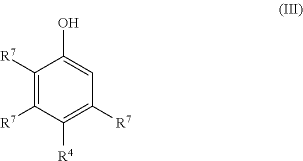

wherein each R⁷ is independently selected from hydrogen, hydroxy, and R⁸, with a secondary amine HNR⁶R⁶ by means of an amino alkylation reaction according to Betti/Mannich in the presence of formaldehyde in a polar solvent, thereby substituting the hydrogen atom in the ortho position to the phenolic OH group, and optionally another substitutable hydrogen atom R⁷ of the phenol derivative of formula (II), by a —CH₂—NR⁶R⁶ moiety (each), resulting in a corresponding Betti base according to formula (IV) or (V):

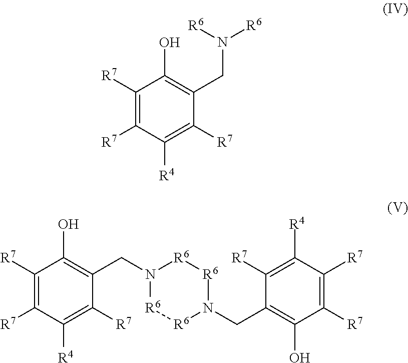

wherein each R⁷ is independently selected from hydrogen, hydroxy, R⁸, and in formula (IV) also from —CH₂—NR⁶R⁶;
2) reacting the (two) phenolic OH group(s), and optionally any further free OH group(s) R⁷, of the respective Betti base of formula (IV) or (V) with a compound according to the formula R¹—X, wherein X represents a leaving group selected from halides and sulfonates, by means of an etherification reaction according to Williamson in the presence of a base, in an organic solvent or without any solvent, resulting in a corresponding ether according to formula (VI) or (VII):

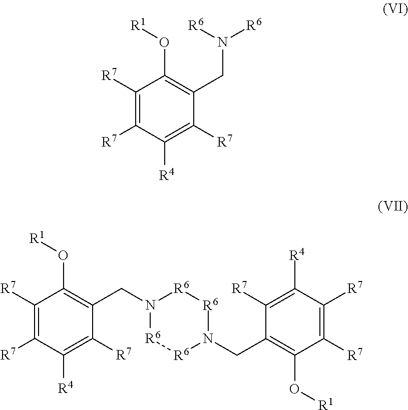

wherein each R⁷ is independently selected from hydrogen, R¹—O—, R⁸, and in formula (VI) also from —CH₂—NR⁶R⁶; and
3) oxidizing any amino groups —NR⁶R⁶ of the respective ether of formula (VI) or (VII) by reaction with an oxidizing agent in water, an organic solvent or a mixture thereof, resulting in the amine N-oxide compound of formula (I) or (II).

In this way, according to the present invention, it is possible to synthesize amine N-oxide compounds from phenol derivatives of the formula (III) by a comparatively simple and inexpensive method that comprises a sequence of known individual reactions. The method according to the invention comprises only three reaction steps, i.e. two fewer than the process for the preparation of the only known aromatic amine N-oxide pDoAO mentioned at the beginning, which is derivable from the combined disclosures of Di Crescenzo et al. and Goracci et al., whereby according to the present invention the compounds according to formula (I) or (II) are sometimes available even in total yields of over 90%. In preferred embodiments, the starting compounds are also readily available products of lignin depolymerization, and the method is carried out in the most environmentally friendly manner possible, especially since both the aminoalkylation according to Betti/Mannich and the etherification of the products according to Williamson are characterized by a high level of atom economy.

In addition, in preferred embodiments of the method, the aminoalkylation according to Betti/Mannich in step 1) is carried out in water and more preferably at room temperature, which avoids the use of solvents and large amounts of energy and, surprisingly, can also increase the yields. However, organic solvents, such as alcohols, e.g. methanol, ethanol or (iso)propanol, or acetonitrile or toluene, can still be used, either instead of or in a mixture with water. However, the use of water as the sole solvent is preferred, provided the solubility of the phenol derivative of the formula (III) allows for this.

The latter is preferably reacted in step 1) with 1.05 and more preferably 1.5 equivalents each of the secondary amine and formaldehyde to ensure complete aminoalkylation at only one position of the aromatic if a monomeric amine N-oxide compound of formula (I) is desired. If a double aminoalkylation is desired, the starting compound used is preferably a phenol derivative of formula (III) which is unsubstituted at the two ortho positions but is very well substituted, for example alkylated, in the para position in order to to direct the two —CH₂—NR⁶R⁶ moieties to be introduced to an ortho position each. If only one ortho hydrogen atom can be substituted, isomeric mixtures of the doubly aminoalkylated Betti base of the formula (IV) are often obtained. However, if a dimeric amine N-oxide compound according to formula (II) is to be prepared, the phenol derivative of the formula (III) is preferably used in a ratio of 2:1 to the secondary diamine (or dimer of the secondary amine).

Alternatively or additionally, in some preferred embodiments of the method, a solid-liquid phase transfer reaction is carried out in the etherification step 2) using a solid base and in the presence of a phase transfer catalyst in order to increase the conversions. Experiments by the inventors using standard protocols for etherification according to Williamson, i.e. as a single-phase in different solvents using a solution of different bases and at different temperatures, also led to the desired compound, but the formation of by-products occurred to varying extents. The reason for this is that Betti bases of formula (IV) with a free OH group in the ortho position tend to decompose to form the respective ortho quinone methide, as shown below:

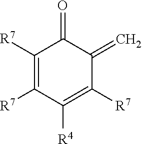

Ortho quinone methides are very reactive and tend to polymerize, which is why aminoalkylation reactions according to Betti using similar starting compounds are completely unknown in the literature.

The fact that both heat and the presence of bases (or acids) promote the decomposition of the Betti bases and the polymerization of the ortho quinone methides is, on the one hand, another reason for the preferred reaction control in step 1) and, on the other hand, that for the development of the solid-liquid phase transfer reaction in step 2) by the present inventors. This is particularly true for the dimeric Betti bases of formula (V), which are more prone to decomposition and polymerization due to the presence of two susceptible moieties.

In particularly preferred embodiments, the base is added in solid form to the respective Betti base of formula (IV) or (V) and the reactants are reacted with one another either in an organic solvent or without any solvent, in particular at room temperature. Particularly preferred is the use of chloride or bromide, in particular bromide, as said leaving group as well as of an anhydrous solvent in order to suppress the formation of by-products. After a series of tests with other solvents, such as acetonitrile, in particular anhydrous 2-methyltetrahydrofuran has proven to be successful as it was able to promote the conversions and selectivities for the desired product the most. A sulfonate, such as mesylate or tosylate, can also be used as said leaving group, although the use of long-chain fatty alcohol sulfonates would be uneconomical since these are already surfactants themselves.

As mentioned before, various aqueous solutions of alkali metal carbonates and hydroxides were initially examined as said base, whereafter, for the reasons above, solid powders of the base were used, with NaOH and especially powdered KOH having proven successful. Tetra-n-butylammonium bromide (TBAB) is particularly preferred as said phase transfer catalyst according to the present invention, although other common catalysts, such as a variety of other quaternary ammonium compounds, can also be used.

In preferred embodiments of the method according to the invention, step 3) is also carried out in a mild, environmentally friendly manner and with the highest quantitative conversion possible, since the purification of amphiphilic molecules is usually quite complex. In some of these preferred embodiments, an aqueous solution of H₂O₂ is used as said oxidizing agent, with methyl formate optionally being added as an additional solvent, the ether of formula (VI) or (VII) being reacted more preferably with 2.5 to 3 equivalents of H₂O₂ to ensure complete conversion.

While organic solvents such as dichloromethane or acetonitrile, sometimes with the use of catalysts, are also suitable, an (e.g. 30%) aqueous solution of H₂O₂ has proven to be an excellent oxidizing agent. To increase the solubility, especially in the case of dimeric amine N-oxides, small amounts of organic solvent can be added, for which purpose methyl formate is preferred according to the invention.

In a second aspect, the present invention also provides the amine N-oxide compounds according to formula (I) or (II), prepared by the method according to the first aspect:

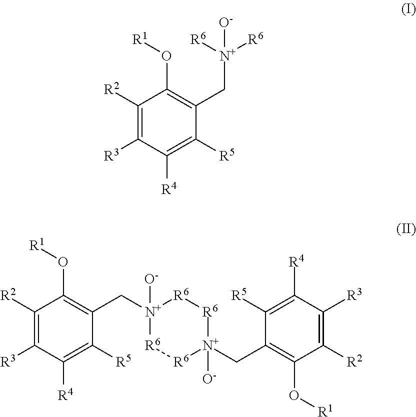

wherein R¹ to R⁶ each are as defined above.

Such amine N-oxide monomers according to formula (I) or corresponding dimers according to formula (II) can not only be produced in a relatively simple and environmentally friendly manner from readily available lignin degradation products, but are also excellently suitable as surfactants. Due to the high hydrophilicity of the N-oxide group(s) and the hydrophobicity of the aromatic(s), even a single-digit number of carbon atoms in the radicals R¹ to R⁵ is sufficient to impart the compounds with the required amphiphilicity.

Preferably, however, the number of carbon atoms in the radicals R¹ to R⁵ is at least 9 carbon atoms. This is particularly preferable with regard to hydrophobicity if two or more amine N-oxide moieties —CH₂—N⁺(O⁻)R⁶R⁶ are bound to the aromatic. But also the fact that the main products of the depolymerization of lignin include not only the derivatives of guaiacol and syringol mentioned at the beginning but also those of catechol, in particular derivatives substituted once or twice with lower alkyl and/or lower alkoxy, simplifies the synthesis of amine N-oxide compounds with at least 9 carbon atoms, since the respective free phenolic OH groups only need to be etherified with easily available and biodegradable fatty alkyl radicals.

Since, on the one hand, fatty alcohols occur in nature in both saturated and unsaturated form, i.e. with one or more C═C double bonds, and, on the other hand, as mentioned at the beginning, the lignin degradation products can also have more than one aromatic ring, but also non-aromatic rings (e.g. dioxolane) as substituents, the definition of R¹ to R⁵ according to the present invention comprises both saturated and unsaturated as well as cyclic radicals.

The fact that, in addition to the amine N-oxide monomers according to formula (I), also dimers according to formula (II) are part of the present invention is due to the synthesis beginning with an aminoalkylation reaction according to Betti/Mannich, which proceeds with secondary mono- and diamines in an analogous manner, which was explained in more detail in relation to the first aspect of the invention and is proven by the later examples.

The lower and upper limits for the number of carbon atoms in the radicals R¹ to R⁵ refers to the preferred use of fatty alkyl radicals for the etherification of free phenolic OH groups in the starting products, for the chain length of which literature specifies between 4 to 6 as the lower limit and between 22 and 26 as the upper limit.

According to the invention, a maximum length of 18 carbon atoms is preferred for the fatty alkyl radicals introduced in the course of the synthesis process by means of etherification or of 4 carbon atoms, respectively, for the alkyl or alkoxy or optionally also alkylthio radicals already bound to the aromatic in the starting material, which particularly applies for the radical R⁴ in the para position to the ether group R¹—O—.

The option that some carbon atoms may be replaced by oxygen or sulfur also refers primarily to the substitution pattern of the starting compounds, preferably obtained by lignin depolymerization, which, as mentioned at the beginning, may have various oxygen-containing functionalities, but sometimes also sulfur analogues thereof. Other heteroatoms, such as halogens or nitrogen, are hardly present in such compounds. While the former would not interfere with the synthesis method according to the first aspect of the invention, nitrogen atoms in the radicals R¹ to R⁵ would most likely also be oxidized to N-oxides in the final oxidation step, which would reduce the hydrophobicity of this section of the compounds according to the invention, especially if the nitrogen atoms were not positioned very close to the aromatic ring. Therefore, for the purposes of the present invention, heteroatoms other than oxygen and sulfur need not be considered.

However, the option that one or more of the radicals R², R³ and R⁵ can also represent an amine-N-oxide moiety —CH₂—N⁺(O⁻)R⁶R⁶, refers specifically to the synthesis process, in the first step of which the aromatic ring can also be aminoalkylated at more than one position—which was actually done, as the examples demonstrate.

In some preferred embodiments of the invention, R¹ is therefore C₆-C₂₂ alkyl, more preferably C₈-C₁₈ alkyl.

Alternatively or additionally, in some preferred embodiments, R² is selected from C₁-C₂₂ alkyl, C₁-C₂₂ alkoxy, and —CH₂—N⁺(O⁻)R⁶R⁶, more preferably from C₁-C₁₈ alkoxy and —CH₂—N⁺(O⁻)R⁶R⁶. Alternatively or additionally, in some preferred embodiments, R³ and R⁵ are each selected from hydrogen and —CH₂—N⁺(O⁻)R⁶R⁶, with one of R³ and R⁵ being particularly preferably hydrogen and the other —CH₂—N⁺(O⁻)R⁶R⁶. The option —CH₂—N⁺(O⁻)R⁶R⁶ for R², R³ and R⁵ refers to a multiple aminoalkylation of the aromatic, as mentioned above.

Alternatively or additionally, in some preferred embodiments, R⁴ is selected from hydrogen, C₁-C₄ alkyl, and C₁-C₄ alkoxy, more preferably from hydrogen and C₁-C₄ alkyl, and most preferably it is C₁-C₄ alkyl.

In some particularly preferred embodiments—after only simple aminoalkylation in the synthesis process—the following applies:
R¹ is C₈-C₁₈ alkyl;
R² is C₁-C₁₈ alkoxy;
R³ and R⁵ each are hydrogen; and
R⁴ is C₁-C₄ alkyl, most preferably ethyl or propyl.

If a derivative of catechol with two vicinal phenolic OH groups was used as the starting substance in the synthesis process, R² is particularly preferably C₁-C₁₈ alkoxy; otherwise, however, for example when using a derivative of guaiacol or syringol, R² most preferably is methoxy. In the latter case, when using Syringol, R⁵ is methoxy, too.

However, in some particularly preferred embodiments—after double aminoalkylation in the synthesis process—the following applies:
R¹ is C₈-C₁₈ alkyl;
R² is —CH₂—N⁺(O⁻)R⁶R⁶;
R³ and R⁵ each are hydrogen; and
R⁴ is C₁-C₄ alkyl, most preferably ethyl oder propyl.

With regard to the radicals R⁶, which can generally comprise up to six carbon and optionally heteroatoms (O, S or especially N), in some preferred embodiments of the second aspect of the present invention, they are each independently selected from methyl, ethyl and dimethylaminoethyl.

Alternatively or additionally, in some preferred embodiments, two radicals R⁶ bound to the same nitrogen atom are connected to one another, forming one of the following groups together with the nitrogen atom:

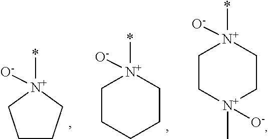

wherein the asterisks designate the respective connections to the aromatic ring.

In some particularly preferred embodiments, all radicals R⁶ are methyl and one or both methyl group(s) of a moiety —N⁺(O⁻)(CH₃)₂ is/are linked to one or both methyl group(s) of such a moiety of another molecule of formula (I), thus forming a bridge having the structure

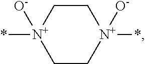

wherein the dashed line designates an optional bond between the two methyl groups and the asterisks designate the connections of the bridge to the two aromatic rings, thereby forming a dimer of the amine N-oxide compound according to formula (II).

In particular, the amine N-oxide compound according to the second aspect of the present invention is selected from the following compounds:

### N,N-dimethyl-1-(5-ethyl-3-methoxy-2-octyloxyphenyl)methanamine N-oxide (1)

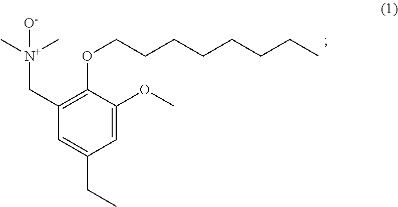 [SMILES: CCCCCCCCOc1c(C[N+](C)(C)[O-])cc(CC)cc1OC]

### N,N-dimethyl-1-(2-decyloxy-5-ethyl-3-methoxyphenyl)methanamine N-oxide (2)

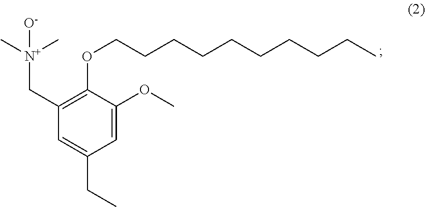 [SMILES: CCCCCCCCCCOc1c(C[N+](C)(C)[O-])cc(CC)cc1OC]

### N,N-dimethyl-1-(2-dodecyloxy-5-ethyl-3-methoxyphenyl)methanamine N-oxide (3)

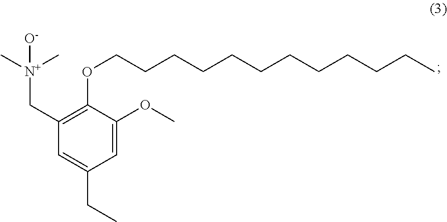 [SMILES: CCCCCCCCCCCCOc1c(C[N+](C)(C)[O-])cc(CC)cc1OC]

### N,N-dimethyl-1-(5-ethyl-3-methoxy-2-tetradecyloxyphenyl)methanamine N-oxide (4)

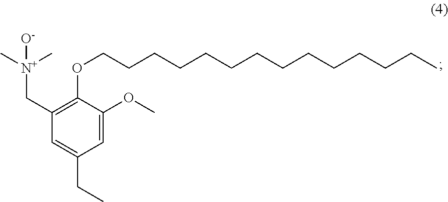 [SMILES: CCCCCCCCCCCCCCOc1c(C[N+](C)(C)[O-])cc(CC)cc1OC]

### N,N-dimethyl-1-(5-ethyl-2-hexadecyloxy-3-methoxyphenyl)methanamine N-oxide (5)

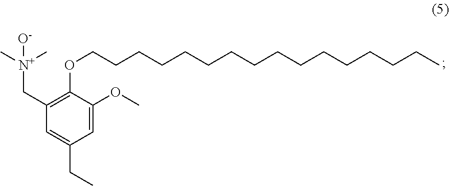 [SMILES: CCCCCCCCCCCCCCCCOc1c(C[N+](C)(C)[O-])cc(CC)cc1OC]

### N,N-dimethyl-1-(5-ethyl-3-methoxy-2-octadecyloxyphenyl)methanamine N-oxide

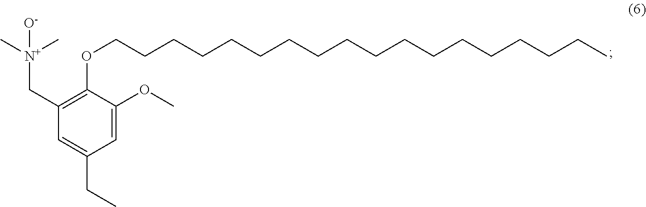 [SMILES: CCCCCCCCCCCCCCCCCCOc1c(C[N+](C)(C)[O-])cc(CC)cc1OC]

### 1-(2-dodecyloxy-5-ethyl-3-methoxybenzyl)pyrrolidine-1-oxide (7)

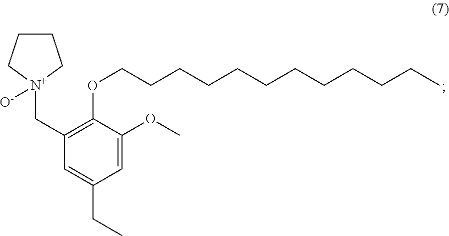 [SMILES: CCCCCCCCCCCCOc1c(C[N+]2([O-])CCCC2)cc(CC)cc1OC]

### 1-(2-dodecyloxy-5-ethyl-3-methoxybenzyl)piperidine-1-oxide (8)

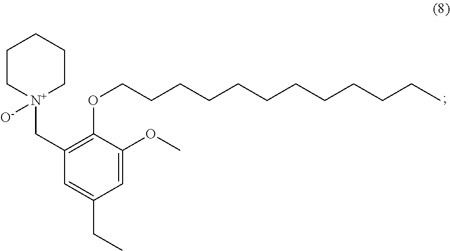 [SMILES: CCCCCCCCCCCCOc1c(C[N+]2([O-])CCCCC2)cc(CC)cc1OC]

### 1-(2-dodecyloxy-5-ethyl-3-methoxybenzyl)-4-methylpiperazine-1,4-dioxide (9)

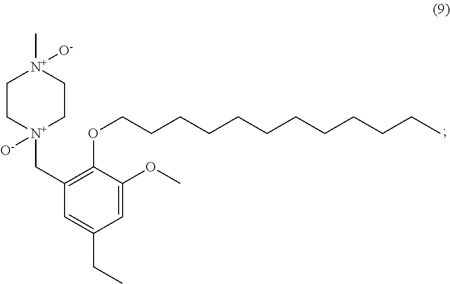 [SMILES: CCCCCCCCCCCCOc1c(C[N+]2([O-])CC[N+](C)([O-])CC2)cc(CC)cc1OC]

### N,N-dimethyl-N′-(2-dodecyloxy-5-ethyl-3-methoxybenzyl)-N′-methylethane-1,2-diamine di-N-oxide (10)

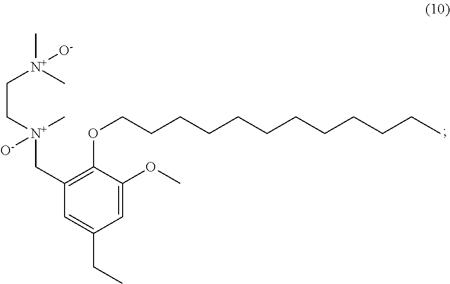 [SMILES: CCCCCCCCCCCCOc1c(C[N+](C)([O-])CC[N+](C)(C)[O-])cc(CC)cc1OC]

### 1,1′-(2,3-dioctyloxy-5-ethyl-1,4-phenylene)-bis(N,N-dimethylmethanamine N-oxide) and 1,1′-(2,3-dioctyloxy-5-ethyl-1,6-phenylene)-bis(N,N-dimethylmethanamine N-oxide) (11)

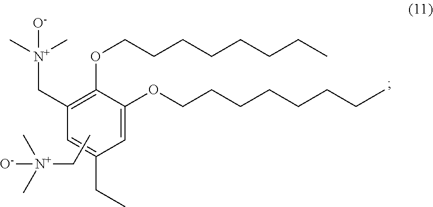

### 1,1′-(5-ethyl-2-octyloxy-1,3-phenylene)-bis(N,N-dimethylmethanamine N-oxide) (12)

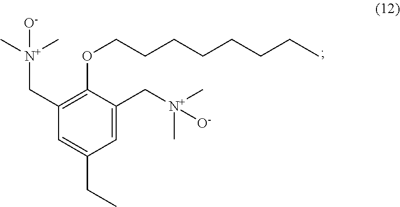 [SMILES: CCCCCCCCOc1c(C[N+](C)(C)[O-])cc(CC)cc1C[N+](C)(C)[O-]]

### 1,1′-(2-dodecyloxy-5-ethyl-1,3-phenylene)-bis(N,N-dimethylmethanamine N-oxide) (13)

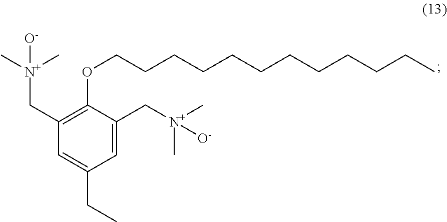 [SMILES: CCCCCCCCCCCCOc1c(C[N+](C)(C)[O-])cc(CC)cc1C[N+](C)(C)[O-]]

### 1,1′-(5-ethyl-2-hexadecyloxy-1,3-phenylene)-bis(N N-dimethylmethanamine N-oxide) (14)

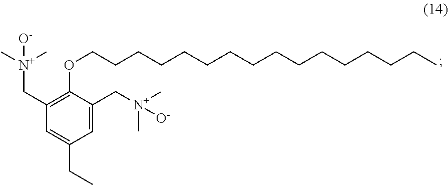 [SMILES: CCCCCCCCCCCCCCCCOc1c(C[N+](C)(C)[O-])cc(CC)cc1C[N+](C)(C)[O-]]

### 1,1′-(5-ethyl-2-octadecyloxy-1,3-phenylene)-bis(N,N-dimethylmethanamine N-oxide) (15)

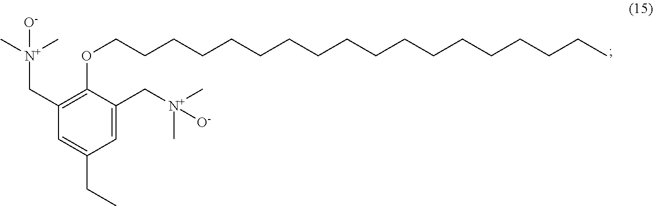 [SMILES: CCCCCCCCCCCCCCCCCCOc1c(C[N+](C)(C)[O-])cc(CC)cc1C[N+](C)(C)[O-]]

### 1,4-bis(5-ethyl-3-methoxy-2-octyloxybenzyl)piperazine-1,4-dioxide (16)

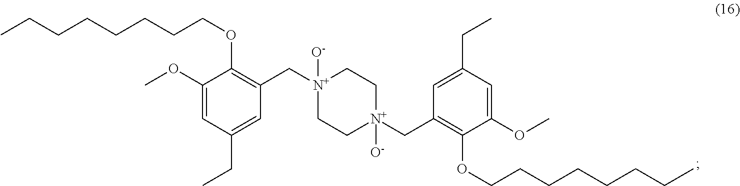 [SMILES: CCCCCCCCOc1c(C[N+]2([O-])CC[N+]([O-])(Cc3cc(CC)cc(OC)c3OCCCCCCCC)CC2)cc(CC)cc1OC]

### 1,4-bis(2-dodecyloxy-5-ethyl-3-methoxybenzyl)piperazine-1,4-dioxide (17)

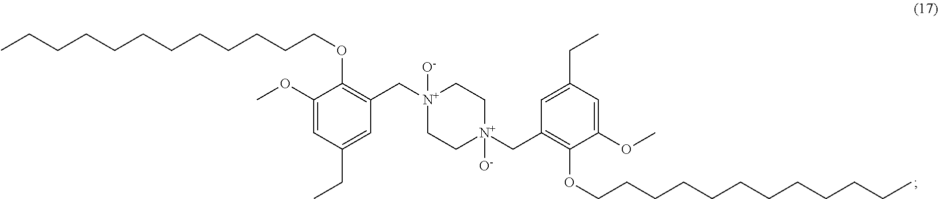 [SMILES: CCCCCCCCCCCCOc1c(C[N+]2([O-])CC[N+]([O-])(Cc3cc(CC)cc(OC)c3OCCCCCCCCCCCC)CC2)cc(CC)cc1OC]

### N,N′-bis(5-ethyl-3-methoxy-2-octyloxybenzyl)-N,N′-dimethylethane-1,2-diamine di-N-oxide (18)

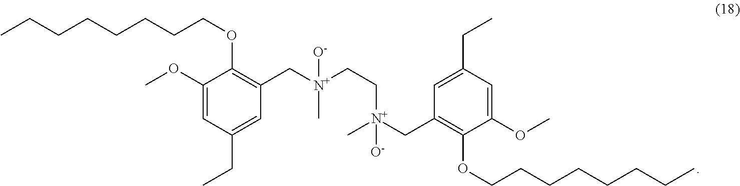 [SMILES: CCCCCCCCOc1c(C[N+](C)([O-])CC[N+](C)([O-])Cc2cc(CC)cc(OC)c2OCCCCCCCC)cc(CC)cc1OC]

And in a third aspect, the present invention relates to the use of the novel amine N-oxide compounds according to formula (I) or (II), in which the total number of carbon atoms of the radicals R¹ to R⁵ should be at least 9, as surfactants.

### BRIEF DESCRIPTION OF THE FIGURE

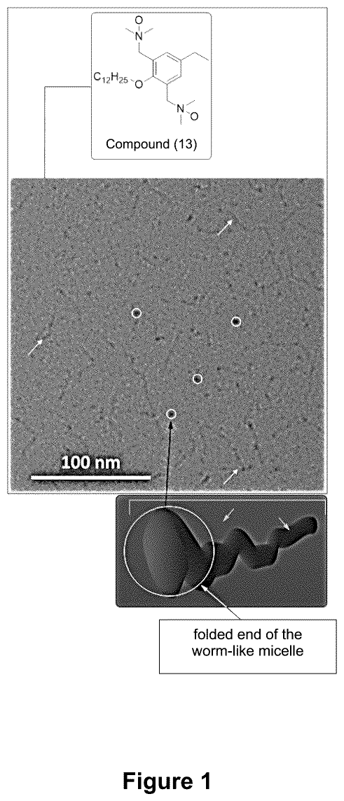

The only FIG. 1 shows a cryogenic electron microscope image of an aqueous solution of the amine N-oxide compound (13) from Example 13 together with a schematic representation of the micelles to be observed therein.

### EXAMPLES

The present invention will be described in more detail below by way of examples, which, however, should not be construed as limiting the scope of protection. For illustration purposes, two representative model compounds for the common lignin depolymerization products preferred as starting substances in the method according to the invention were used.

For this purpose, one phenol and one diphenol derivative, namely 4-ethylguaiacol (4-ethyl-2-methoxyphenol) and 4-ethylcatechol (1,2-dihydroxy-4-ethylbenzene), were reacted to the amine N-oxide compounds according to the invention:

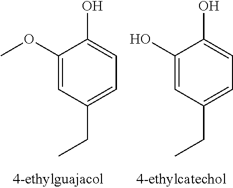

These were first aminomethylated, once or twice, with various secondary amines and diamines in the presence of formaldehyde to obtain corresponding Betti bases, which were then etherified once or twice with a series of fatty alkyl halides and finally oxidized to the amine N-oxides.

### Example 1

### Preparation of N,N-dimethyl-1-(5-ethyl-3-methoxy-2-octyloxyphenyl)methanamine N-oxide (1)

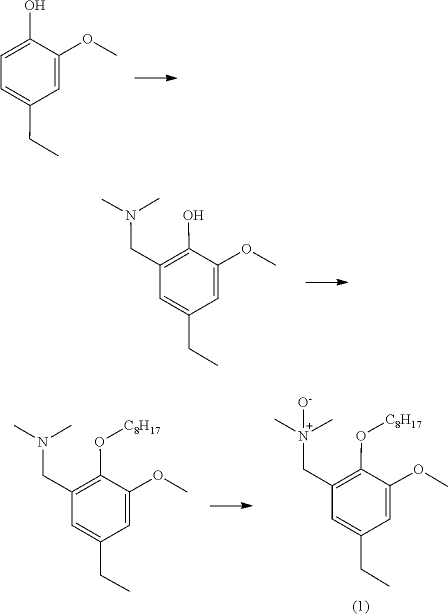

### Step 1:

### Variant 1.1:

An aqueous solution of 4-ethylguaiacol (15.20 g, 100 mmol) was added dropwise to a 40 wt % aqueous solution of dimethylamine (6.76 g, 150 mmol) in an iced water bath within 15 minutes with constant stirring. Paraformaldehyde (4.50 g, 150 mmol) was added in aliquots of 0.5 g every 10 min and stirred for 3 h in an iced water bath and then for 9 h at room temperature. The volatile components were then removed on a rotary evaporator at room temperature, whereafter the water was removed at 50° C. in vacuo and the residue was completely dried in a vacuum desiccator. The aminomethylated intermediate product, 2-dimethylaminomethyl-4-ethyl-6-methoxyphenol, was obtained as a viscous, yellow oil (yield: 20.83 g; 99.5% of theory).

### Variant 1.2:

An aqueous solution of 4-ethylguaiacol (15.20 g, 100 mmol) was added dropwise to a 40 wt % aqueous solution of dimethylamine (4.56 g, 101 mmol) in an iced water bath within 15 minutes with constant stirring. Paraformaldehyde (4.50 g, 150 mmol) was added in aliquots of 0.5 g every 10 min and stirred for 3 h in an iced water bath and then for 72 h at room temperature. The reaction mixture was then extracted 5 times with 25 ml of petroleum ether (bp.: 40-60° C.), and the combined organic phases were concentrated in vacuo on a rotary evaporator, whereafter the residue was completely dried in a vacuum desiccator. The aminomethylated intermediate, 2-dimethylaminomethyl-4-ethyl-6-methoxyphenol, was obtained as a viscous, yellowish oil (yield: 19.43 g; 93.0% of theory).

### Step 2:

### Variant 2.1:

2-Dimethylaminomethyl-4-ethyl-6-methoxyphenol (1.05 g, 5 mmol), 1-bromooctane (0.95 g, 4.9 mmol) and tetrabutylammonium bromide (TBAB) (0.16 g, 0.5 mmol) as a catalyst in 10 ml of 2-methyltetrahydrofuran (2-MeTHF) as solvent were stirred vigorously at room temperature until a homogeneous solution was obtained, whereafter solid powdered KOH (0.56 g, 10 mmol) was added and stirred at room temperature for 8 h. The inorganic solid was then centrifuged off and washed 3 times with 10 ml of diethyl ether. The combined organic phases were concentrated on a rotary evaporator, the residue was redissolved in 45 ml of petroleum ether and washed 4 times with 5 ml of water. The organic phase was then concentrated on a rotary evaporator and the residue was completely dried in a vacuum desiccator. The etherified intermediate, N,N-dimethyl-1-(5-ethyl-3-methoxy-2-octyloxyphenyl)methanamine, was obtained as a viscous, yellow oil (yield: 1.45 g; 92.3% of theory).

### Variant 2.2:

2-Dimethylaminomethyl-4-ethyl-6-methoxyphenol (1.05 g, 5 mmol), 1-bromooctane (1.06 g, 5.5 mmol) and tetrabutylammonium bromide (TBAB) (0.16 g, 0.5 mmol) in 10 ml 2-MeTHF were stirred vigorously at room temperature until a homogeneous solution was obtained, whereafter solid powdered KOH (0.56 g, 10 mmol) was added and stirred at room temperature for 8 h. Then 25 ml Et₂O and 5 ml H₂O were added, and the aqueous phase was extracted 3 times with 10 ml Et₂O. The combined organic phases were concentrated on a rotary evaporator and the residue was stored over 5 g of silica gel. The residue was purified using flash chromatography on a filter column, the etherified intermediate product, at which N,N-dimethyl-1-(5-ethyl-3-methoxy-2-octyloxyphenyl)methanamine eluted together with Et₂O and, after evaporation of the ether, was obtained as a viscous, yellow oil (yield: 1.45 g; 92.9% of theory).

### Step 3:

N,N-Dimethyl-1-(5-ethyl-3-methoxy-2-octyloxyphenyl)methanamine (0.96 g, 3 mmol) was charged and 3 equivalents of a 30 wt % aqueous solution of H₂O₂ (9 mmol) were added at once. The turbid reaction mixture was stirred at room temperature overnight or until it appeared clear and homogeneous, indicating complete consumption of the starting material. A catalytic amount of active carbon or MnO₂ was then added and the mixture was stirred for 24 h, or oven-dried Na₂CO₃ (1.06 g, 10 mmol) in 5 ml of ethanol was added and the mixture was stirred for 30 min to decompose the excess H₂O₂. The solid precipitate was then centrifuged off and washed 3 times with abs. EtOH. The combined organic phases were filtered through a 0.2 μm syringe filter and then concentrated in vacuo on a rotary evaporator, mixed with hexane and concentrated again to completely remove the EtOH. The residue was then completely dried in vacuo, giving the title compound (1) as a clear, yellow oil (yield: 0.98 g; 97.3% of theory).

¹H NMR: δ_{H} (600 MHz, Chloroform-d) 6.90 (d, J=2.0 Hz, 1H, C3), 6.81 (d, J=2.0 Hz, 1H, C5), 4.54 (s, 2H, C12), 3.94 (t, J=6.9, 6.9 Hz, 2H, C17), 3.86 (s, 3H, C11), 3.18 (s, 6H, C14, C15), 2.61 (q, J=7.6, 7.6, 7.6 Hz, 2H, C7), 1.77 (p, J=7.1, 7.1, 7.1, 7.1 Hz, 2H, C18), 1.41 (p, J=7.1, 7.1, 6.8, 6.8 Hz, 2H, C19), 1.35-1.19 (m, 11H, C8, C20-23), 0.88 (t, J=6.9, 6.9 Hz, 3H, C24). ¹³C NMR: (151 MHz, CDCl₃) δ_{C} 152.7 (C6), 146.0 (C1), 140.4 (C4), 124.6 (C3), 123.9 (C2), 114.0 (C5), 74.0 (C17), 70.5 (C12), 57.4 (C14, C15), 55.9 (C11), 32.0 (C18), 30.4 (C19), 29.5 (C20), 29.4 (C21), 28.7 (C7), 26.1 (C22), 22.8 (C23), 15.7 (C8), 14.2 (C24); HRMS: (ESI⁺, m/z) calculated for C₂₀H₃₆NO₃ [M+H]⁺: 338.26929; found: 338.26292.

### Example 2

### Preparation of N,N-dimethyl-1-(2-decyloxy-5-ethyl-3-methoxyphenyl)methanamine N-oxide (2)

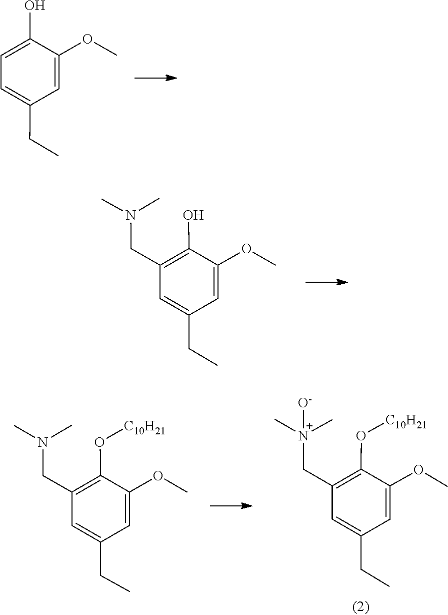

### Step 1:

The synthesis and the product were identical to Example 1.

### Step 2:

The reaction was carried out in an analogous manner to that in Example 1, except that 1-bromodecane was used instead of 1-bromooctane, giving N,N-dimethyl-1-(2-decyloxy-5-ethyl-3-methoxyphenyl)methanamine as a viscous, yellow oil.

### Variant 2.1:

Yield: 1.50 g; 87.6% of theory

### Variant 2.2:

Yield: 1.63 g; 93.2% of theory

### Step 3:

The reaction was carried out in an analogous manner to that in Example 1, except that only 2 mmol of N,N-dimethyl-1-(2-decyloxy-5-ethyl-3-methoxyphenyl)methanamine were used, giving the title compound (2) as a clear, yellow oil (yield: 0.70 g; 96.1% of theory).

¹H NMR: δ_{H} (600 MHz, Chloroform-d) 6.89 (d, J=2.0 Hz, 1H, C3), 6.79 (d, J=2.0 Hz, 1H, C5), 4.52 (s, 2H, C17), 3.93 (t, J=6.9, 6.9 Hz, 2H, C18), 3.84 (s, 3H, C11), 3.16 (s, 6H, C14, C15), 2.60 (q, J=7.6, 7.6, 7.6 Hz, 2H, C7), 1.76 (p, J=7.1, 7.1, 7.1, 7.1 Hz, 2H, C18), 1.39 (p, J=7.4, 7.4, 6.9, 6.9 Hz, 2H, C19), 1.34-1.17 (m, 16H, C8, C20-C25), 0.86 (t, J=7.0, 7.0 Hz, 3H, C26). ¹³C NMR: δ_{C} (151 MHz, Chloroform-d) 152.6 (C6), 145.9 (C1), 140.3 (C4), 124.5 (C3), 124.0 (C2), 113.9 (C5), 74.0 (C17), 70.5 (C12), 57.5 (C14, C15), 55.8 (C11), 32.0 (C18), 30.4 (C19), 29.7 (C20, C21), 29.5 (C22), 29.4 (C23), 28.7 (C7), 26.1 (C24), 22.8 (C25), 15.6 (C8), 14.2 (C26). HRMS: (ESI⁺, m/z) calculated for C₂₂H₄₀NO₃ [M+H]⁺: 366.30027; found: 366.30049.

### Example 3

### Preparation of N,N-Dimethyl-1-(2-dodecyloxy-5-ethyl-3-methoxyphenyl)methanamin-N-oxid (3)

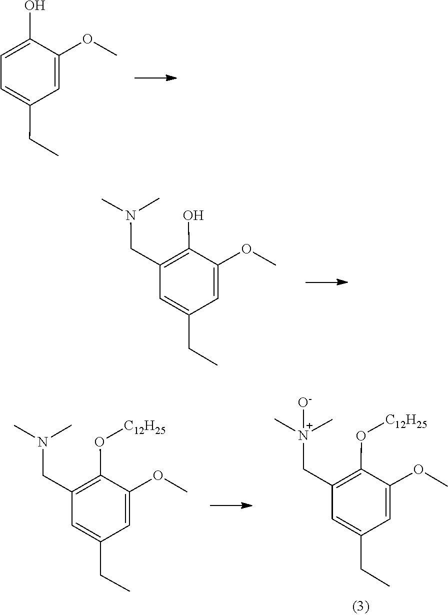

### Step 1:

The synthesis and the product were identical to Example 1.

### Step 2:

The reaction was carried out in an analogous manner to that in Example 1, except that 1-bromododecane was used instead of 1-bromooctane, giving N,N-dimethyl-1-(2-dodecyloxy-5-ethyl-3-methoxyphenyl)methanamine as a viscous, yellow oil.

### Variant 2.1:

Yield: 1.77 g; 96.0% of theory

### Variant 2.2:

Yield: 1.67 g; 88.4% of theory

### Step 3:

The reaction was carried out in an analogous manner to that in Example 1, except that only 2 mmol of N,N-dimethyl-1-(2-dodecyloxy-5-ethyl-3-methoxyphenyl)methanamine were used, giving the title compound (3) as a clear, yellow oil (yield: 0.50 g; 95.2% of theory).

¹H NMR: δ_{H} (600 MHz, Chloroform-d) 6.90 (d, J=2.0 Hz, 1H, C3), 6.81 (d, J=2.0 Hz, 1H, C5), 4.54 (s, 2H, C12), 3.94 (t, J=6.9, 6.9 Hz, 2H, C18), 3.86 (s, 3H, C11), 3.18 (s, 6H C14, C15), 2.61 (q, J=7.6, 7.6, 7.6 Hz, 2H, C7), 1.77 (p, J=7.1, 7.1, 7.1, 7.1 Hz, 2H, C18), 1.41 (p, J=7.3, 7.3, 6.9, 6.9 Hz, 2H, C19), 1.37-1.20 (m, 20H, C8, C20-C27), 0.87 (t, J=6.9, 6.9 Hz, 3H, C28). ¹³C NMR: δ_{C} (151 MHz, Chloroform-d) 152.7 (C6), 146.0 (C1), 140.4 (C4), 124.5 (C3), 123.9 (C2), 114.0 (C5), 74.0 (C17), 70.5 (C12), 57.5 (C14, C15), 55.9 (C11), 32.0 (C18), 30.4 (C19), 29.8 (C20), 29.8, (C21), 29.8 (C22), 29.7 (C23), 29.6 (C24), 29.5 (C25), 28.7 (C7), 26.1 (C26), 22.8 (C27), 15.7 (C8), 14.3 (C28); HRMS: (ESI⁺, m/z) calculated for C₂₄H₄₄NO₃ [M+H]⁺: 394.33157; found: 394.33176.

### Example 4

### Preparation of N,N-dimethyl-1-(5-ethyl-3-methoxy-2-tetradecyloxyphenyl)methanamine N-oxide (4)

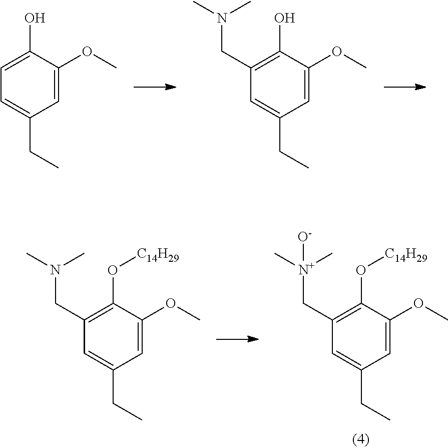

### Step 1:

The synthesis and the product were identical to Example 1.

### Step 2:

The reaction was carried out in an analogous manner to that in Example 1, except that 1-bromotetradecane was used instead of 1-bromooctane, giving N,N-dimethyl-1-(5-ethyl-3-methoxy-2-tetradecyloxyphenyl)methanamine as a viscous, yellow oil.

### Variant 2.1:

Yield: 1.91 g; 96.1% of theory

### Variant 2.2:

Yield: 1.85 g; 91.1% of theory

### Step 3:

The reaction was carried out in an analogous manner to that in Example 1, except that only 2 mmol of N,N-dimethyl-1-(5-ethyl-3-methoxy-2-tetradecyloxyphenyl)methanamine were used, giving the title compound (4) as a clear, yellow oil (yield: 0.80 g; 94.3% of theory).

¹H NMR: δ_{H} (600 MHz, Chloroform-d) 6.89 (d, J=2.0 Hz, 1H, C3), 6.80 (d, J=2.0 Hz, 1H, C5), 4.55 (s, 2H, C12), 3.93 (t, J=6.9, 6.9 Hz, 2H, C17), 3.85 (s, 3H, C11), 3.19 (s, 6H, C14, C15), 2.60 (q, J=7.6, 7.6, 7.6 Hz, 2H, C7), 1.76 (p, J=7.1, 7.1, 7.1, 7.1 Hz, 2H, C18), 1.40 (p, J=7.3, 7.3, 6.9, 6.9 Hz, 2H, C19), 1.34-1.19 (m, 23H, C18, C20-C29), 0.86 (t, J=7.0, 7.0 Hz, 3H, C30). ¹³C-NMR: δ_{C} (151 MHz, Chloroform-d) 152.6 (C6), 145.9 (C1), 140.4 (C4), 124.6 (C3), 123.7 (C2), 114.0 (C5), 74.0 (C17), 70.2 (C12), 57.2 (C14, C15), 55.9 (C11), 32.0 (C18), 30.4 (C19), 29.8 (C20, C21), 29.8 (C22, C23), 29.8 (C24), 29.7 (C25), 29.5 (C26), 29.5 (C27), 28.7 (C7), 26.1 (C28), 22.8 (C29), 15.7 (C8), 14.2 (C30); HRMS: (ESI⁺, m/z) calculated for C₂₆H₄₈NO₃ [M+H]⁺: 422.36287; found: 422.36323.

### Example 5

### Preparation of N,N-dimethyl-1-(5-ethyl-2-hexadecyloxy-3-methoxyphenyl)methanamine N-oxide (5)

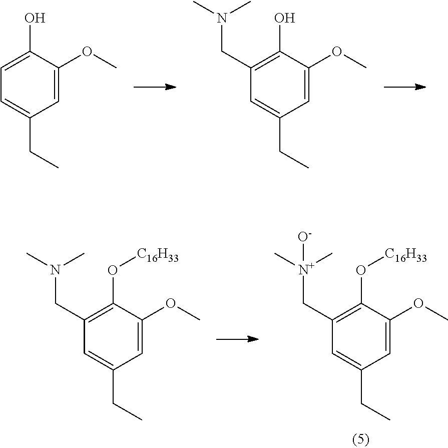

### Step 1:

The synthesis and the product were identical to Example 1.

### Step 2:

The reaction was carried out in an analogous manner to that in Example 1, except that 1-bromohexadecane was used instead of 1-bromooctane, giving N,N-dimethyl-1-(5-ethyl-2-hexadecyloxy-3-methoxyphenyl)methanamine as a viscous, yellow oil.

### Variant 2.1:

Yield: 2.00 g; 94.0% of theory

### Variant 2.2:

Yield: 2.11 g; 97.4% of theory

### Step 3:

The reaction was carried out in an analogous manner to that in Example 1, except that only 2 mmol of N,N-dimethyl-1-(5-ethyl-2-hexadecyloxy-3-methoxyphenyl)methanamine were used, giving the title compound (5) as a clear, yellow oil (yield: 0.87 g; 97.0% of theory).

¹H NMR: δ_{H} (600 MHz, Chloroform-d) 6.90 (d, J=1.9 Hz, 1H, C3), 6.81 (d, J=1.9 Hz, 1H, C5), 4.53 (s, 2H, C11), 3.94 (t, J=6.9, 6.9 Hz, 2H, C17), 3.86 (s, 3H, C11), 3.18 (s, 6H, C14, C15), 2.62 (q, J=7.6, 7.6, 7.6 Hz, 2H, C7), 1.77 (p, J=7.1, 7.1, 7.1, 7.1 Hz, 2H, C18), 1.41 (p, J=7.3, 7.3, 6.9, 6.9 Hz, 2H, C19), 1.36-1.20 (m, 29H, C8, C20-C31), 0.87 (t, J=6.9, 6.9 Hz, 3H, C32). ¹³C NMR: δ_{C} (151 MHz, Chloroform-d) 152.7 (C6), 146.0 (C1), 140.4 (C4), 124.5 (C3), 123.9 (C2), 114.0 (C5), 74.0 (C17), 70.5 (C12), 57.5 (C14, C15), 55.9 (C11), 32.1 (C18), 30.4 (C19), 29.8 (C20), 29.8 (C21, C22), 29.8 (C23, C24), 29.8 (C25), 29.7 (C26), 29.6 (C27), 29.5 (C28), 28.7 (C29) (C7), 26.1 (C30), 22.8 (C31), 15.7 (C8), 14.3 (C32). HRMS: (ESI⁺, m/z) calculated for C₂₈H₅₂NO₃ [M+H]⁺: 450.39417; found: 450.39428.

### Example 6

### Preparation of N,N-dimethyl-1-(5-ethyl-3-methoxy-2-octadecyloxyphenyl)methanamine N-oxide (6)

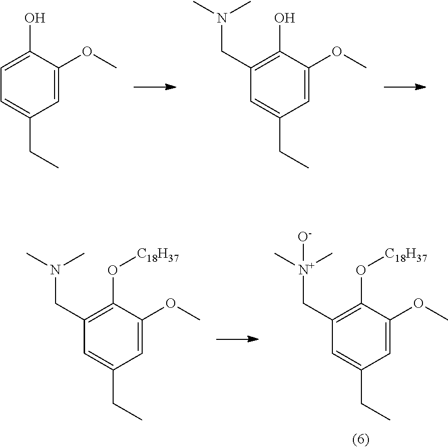

### Step 1:

The synthesis and the product were identical to Example 1.

### Step 2:

The reaction was carried out in an analogous manner to that in Example 1, except that 1-bromooctadecane was used instead of 1-bromooctane, giving N,N-dimethyl-1-(5-ethyl-2-octadecyloxy-3-methoxyphenyl)methanamine as a viscous, yellow oil (Variant 2.1; yield: 2.06 g; 91.0% of theory) or as a white, waxy solid (Variant 2.2; yield: 2.00 g; 86.3% of theory.

### Step 3:

The reaction was carried out in an analogous manner to that in Example 1, except that only 2 mmol of N,N-dimethyl-1-(5-ethyl-2-octadecyloxy-3-methoxyphenyl)methanamine were used and 0.5 ml MeOH were added for enhancing solubility, giving the title compound (6) as a white, waxy solid (yield: 0.916 g; 95.9% of theory).

¹H NMR: δ_{H} (600 MHz, Chloroform-d) 6.91 (d, J=2.0 Hz, 1H, C3), 6.82 (d, J=2.0 Hz, 1H, C5), 4.56 (s, 2H, C12), 3.95 (t, J=6.9, 6.9 Hz, 2H, C17), 3.86 (s, 3H, C11), 3.19 (s, 6H, C14, C15), 2.62 (q, J=7.6, 7.6, 7.6 Hz, 2H, C7), 1.77 (p, J=7.1, 7.1, 7.1, 7.1 Hz, 2H, C18), 1.41 (p, J=7.2, 7.2, 6.9, 6.9 Hz, 2H, C19), 1.36-1.19 (m, 30H, C8, C20-C33), 0.87 (t, J=7.0, 7.0 Hz, 3H, C34). ¹³C NMR: δ_{C} (151 MHz, Chloroform-d) 152.7 (C6), 146.0 (C1), 140.4 (C4), 124.5 (C3), 123.7 (C2), 114.0 (C5), 74.0 (C17), 70.3 (C12), 57.3 (C14, C15), 55.9 (C11), 32.1 (C18), 30.4 (C19), 29.8 (C20, C21, C22, C23), 29.8 (C24, C25, C26, C27), 29.8 (C28), 29.7 (C29), 29.6 (C30), 29.5 (C31), 28.7 (C7), 26.1 (C32), 22.8 (C33), 15.7 (C8), 14.3 (C34); HRMS: (ESI⁺, m/z) calculated for C₃₀H₅₆NO₃ [M+H]⁺: 478.42547; found: 478.42540.

### Example 7

### Preparation of 1-(2-dodecyloxy-5-ethyl-3-methoxybenzyl)pyrrolidine-1-oxide (7)

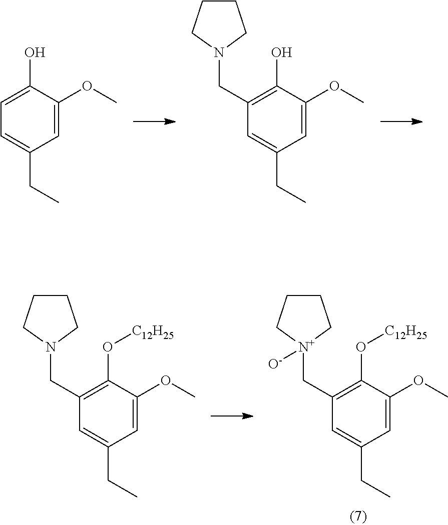

### Step 1:

An aqueous solution of 4-ethylguaiacol (4.56 g, 30 mmol) was added dropwise to an aqueous solution of pyrrolidine (2.21 g, 31 mmol) in an iced water bath within 15 minutes with constant stirring. Paraformaldehyde (1.35 g, 45 mmol) was added in aliquots of 0.5 g every 10 min and stirred for 3 h in an iced water bath and then for 72 h at room temperature. The reaction mixture was then extracted 5 times with 25 ml of petroleum ether, and the combined organic phases were concentrated in vacuo on a rotary evaporator, whereafter the residue was completely dried in a vacuum desiccator. The aminomethylated intermediate, 4-ethyl-6-methoxy-2-(pyrrolidinomethyl)phenol, was obtained as a viscous, yellowish oil (yield: 6.50 g; 92.1% of theory).

### Step 2:

The reaction was carried out in an analogous manner to that in Example 1, Variant 2.1, except that 1-bromododecane was used instead of 1-bromooctane, giving 1-(2-dodecyloxy-5-ethyl-3-methoxybenzyl)pyrrolidine as a viscous, yellowish oil (yield: 1.93 g; 97.6% of theory).

### Step 3:

The reaction was carried out in an analogous manner to that in Example 1, except that only 2 mmol of 1-(2-dodecyloxy-5-ethyl-3-methoxybenzyl)pyrrolidine were used, giving the title compound (7) as a clear, yellow oil (yield: 0.80 g; 95.5% of theory).

¹H NMR: δ_{H} (300 MHz, Chloroform-d) 6.94 (d, J=2.0 Hz, 1H, 2), 6.76 (d, J=2.0 Hz, 1H, 4), 4.64 (s, 2H, 12), 3.91 (t, J=6.9 Hz, 2H, 19), 3.83 (s, 3H, 9), 3.50-3.21 (m, 4H, 14′, 17′), 2.58 (q, J=7.6 Hz, 2H, 10), 2.49-2.34 (m, 2H, 20), 1.87-1.69 (m, 4H, 15″, 16″), 1.37-1.15 (m, 21H, 11, 21, 22, 23, 24, 25, 26, 27, 28, 29), 0.85 (t, J=6.9 Hz, 3H, 30). ¹³C NMR: δ_{C} (75 MHz, Chloroform-d) ¹³C-NMR: δ_{C} (75 MHz, Chloroform-d) 152.5 (5), 145.7 (6), 140.2 (3), 125.1 (2), 124.3 (1), 113.4 (4), 73.9 (19), 65.5 (14, 17), 65.5, (12), 55.8 (9), 32.0 (20), 30.3 (21), 29.7 (22, 23), 29.7 (24, 25), 29.7 (26), 29.5 (27), 29.4 (28), 28.6 (10), 26.1, 22.8, (15, 16), 21.3 (29), 15.6 (11), 14.2 (30); HRMS: (ESI⁺, m/z) calculated for C₂₆H₄₆NO₃ [M+H]⁺: 420.34777; found: 420.347257.

### Example 8

### Preparation of 1-(2-dodecyloxy-5-ethyl-3-methoxybenzyl)piperidine-1-oxide (8)

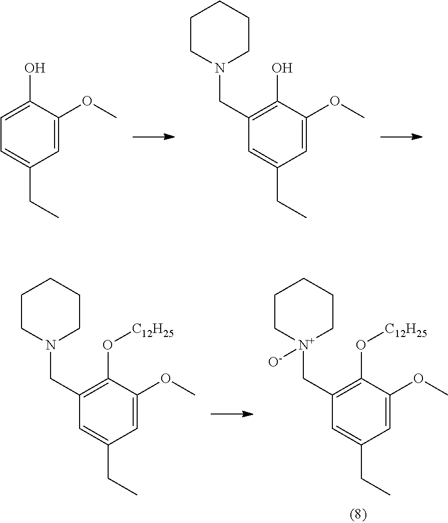

### Step 1:

The reaction was carried out in an analogous manner to that in Example 7, except that piperidine (2.64 g, 31 mmol) was used instead of pyrrolidine, giving 4-ethyl-6-methoxy-2-(piperidinomethyl)phenol as a yellowish oil (yield: 7.06 g; 94.4% of theory).

### Step 2:

The reaction was carried out in an analogous manner to that in Example 1, Variant 2.1, except that 10 mmol of 4-ethyl-6-methoxy-2-(piperidinomethyl)phenol and 1-bromododecane instead of 1-bromooctane were used, giving 1-(2-dodecyloxy-5-ethyl-3-methoxybenzyl)piperidine as a yellowish oil (yield: 3.93 g; 94.1% of theory).

### Step 3:

The reaction was carried out in an analogous manner to that in Example 1, except that 5 mmol of 1-(2-dodecyloxy-5-ethyl-3-methoxybenzyl)piperidine were used, giving the title compound (8) as a clear, yellow oil (yield: 2.10 g; 96.8% of theory).

¹H NMR: δ_{H} (300 MHz, Chloroform-d) 6.99 (d, J=2.0 Hz, 1H), 6.79 (d, J=2.0 Hz, 1H), 4.50 (s, 2H), 3.92 (t, J=1 6.9 Hz, 2H), 3.85 (s, 3H), 3.29 (d, J=11.8 Hz, 2H), 3.04 (td, J=12.2, 3.1 Hz, 2H), 2.61 (q, J=7.6 Hz, 2H), 2.47-2.26 (m, 2H), 1.85-1.02 (m, 28H), 0.87 (t, J=7.0, 6.2 Hz, 3H). ¹³C NMR: δ_{C} (75 MHz, Chloroform-d) 152.4, 146.0, 140.1, 125.3, 123.5, 113.5, 74.0, 70.6, 63.7, 55.8, 32.0, 30.4, 29.8, 29.8, 29.8, 29.6, 29.5, 28.7, 26.2, 22.8, 22.0, 20.6, 15.6, 14.2. HRMS: (ESI⁺, m/z) calculated for C₂₇H₄₈NO₃ [M+H]⁺: 434.36342; found: 434.362833.

### Example 9

### Preparation of 1-(2-dodecyloxy-5-ethyl-3-methoxybenzyl)-4-methylpiperazine-1, 4-dioxide (9)

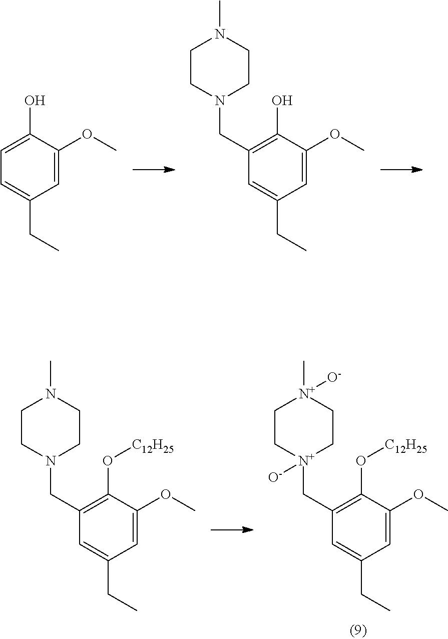

### Step 1:

An aqueous solution of 4-ethylguaiacol (3.04 g, 20 mmol) was added dropwise to an aqueous solution (5 ml) of 1-methylpiperazine (2.64 g, 30 mmol) in an iced water bath within 15 minutes with constant stirring. A 37 wt % aqueous solution of paraformaldehyde (0.90 g, 30 mmol) was added in aliquots of 0.1 g every 10 min and stirred for 3 h in an iced water bath and then for 9 h at room temperature. The precipitated solid was then centrifuged off and re-dissolved in 45 ml of Et₂O. The solution was washed 5 times with 5 ml of water and then concentrated in vacuo on a rotary evaporator, giving the aminomethylated intermediate, 4-ethyl-6-methoxy-2-(4-methylpiperazinomethyl)phenol, as a white powder (yield: 1.65 g; 31.1% of theory).

### Step 2:

The reaction was carried out in an analogous manner to that in Example 1, Variant 2.2, except that 1-bromododecane was used instead of 1-bromooctane, giving 1-(2-dodecyloxy-5-ethyl-3-methoxybenzyl)-4-methylpiperazine as an off-white powder (yield: 0.28 g; 12.9% of theory).

### Step 3:

The reaction was carried out in an analogous manner to that in Example 1, except that 0.125 mmol of 1-(2-dodecyloxy-5-ethyl-3-methoxybenzyl)-4-methylpiperazine were used, giving the title compound (9) as an off-white powder (yield: 0.058 g; 99.9% of theory).

¹H NMR: δ_{H} (300 MHz, CDCl₃) 6.97 (d, J=2.0 Hz, 1H), 6.79 (d, J=2.0 Hz, 1H), 4.46 (s, 2H), 4.39-4.18 (m, 4H), 3.95 (t, J=7.0, 7.0 Hz, 2H), 3.83 (s, 3H), 3.25 (s, 3H), 3.01 (dd, J=15.4, 9.6 Hz, 4H), 2.60 (q, J=7.6, 7.6, 7.6 Hz, 2H), 1.81-1.68 (m, 2H), 1.45-1.13 (m, 22H), 0.85 (d, J=6.8 Hz, 3H). ¹³C NMR: δ_{C} (75 MHz, CDCl₃) 152.2, 145.8, 140.3, 125.4, 121.6, 114.4, 74.0, 69.3, 60.0, 59.1, 57.8, 55.8, 32.0, 29.9, 29.8, 29.8, 29.7, 29.5, 29.4, 28.6, 25.8, 22.8, 15.5, 14.2. HRMS: (ESI⁺, m/z) calculated for C₂₇H₄₈N₂O₄ [M+H]⁺: 465.368684 found: 465.367767.

### Example 10

### Preparation of N,N-dimethyl-N′-(2-dodecyloxy-5-ethyl-3-methoxybenzyl)-N′-methylethane-1,2-diamine di-N-oxide (10)

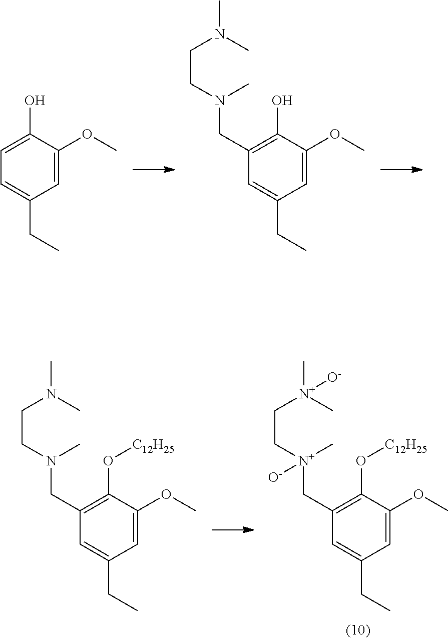

### Step 1:

The reaction was carried out in an analogous manner to that in Example 7, except that N,N,N′-trimethylethane-1,2-diamine (3.07 g, 30 mmol) was used instead of pyrrolidine, giving 2-(2-dimethylaminoethyl)aminomethyl-4-ethyl-6-methoxyphenol as an off-white powder (yield: 4.59 g; 86.3% of theory).

### Step 2:

The reaction was carried out in an analogous manner to that in Example 1, Variant 2.2, except that 1-bromododecane was used instead of 1-bromooctane, giving N,N-dimethyl-N′-(2-dodecyloxy-5-ethyl-3-methoxybenzyl)-N′-methylethane-1,2-diamine as an off-white powder (yield: 1.17 g; 53.8% of theory).

### Step 3:

The reaction was carried out in an analogous manner to that in Example 1, except that 1 mmol of N,N-dimethyl-N′-(2-dodecyloxy-5-ethyl-3-methoxybenzyl)-N′-methylethane-1,2-diamine was used, giving the title compound (10) as as an off-white powder (yield: 0.97 g; 69.0% of theory).

¹H NMR: δ_{H} (300 MHz, CDCl₃) 6.95 (d, J=2.0 Hz, 1H), 6.80 (d, J=2.0 Hz, 1H), 4.49 (d, J=12.4 Hz, 1H), 4.36 (d, J=12.4 Hz, 1H), 4.07-3.92 (m, 2H), 3.91-3.79 (m, 3+3H), 3.67 (q, J=7.0, 7.0, 7.0 Hz, 1H), 3.33-3.17 (m, 6H), 3.04 (s, 3H), 2.70-2.56 (m, 2H), 1.75 (dd, J=10.8, 4.7 Hz, 2H), 1.45-1.35 (m, 2H), 1.35-1.17 (m, 28H), 0.85 (t, J=6.9 Hz, 3H). ¹³C NMR: δ_{C} (75 MHz, CDCl₃) 152.6, 146.0, 140.4, 124.2, 114.1, 74.0, 64.6, 55.9, 32.0, 30.4, 29.8, 29.7, 29.6, 29.5, 28.7, 26.1, 22.8, 15.7, 14.2.

### Example 11

### Preparation of 1,1′-(2,3-dioctyloxy-5-ethyl-1,4-phenylene)-bis(N,N-dimethylmethanamine N-oxide) and 1,1′-(2,3-dioctyloxy-5-ethyl-1,6-phenylene)-bis(N,N-dimethylmethanamine N-oxide) (11)

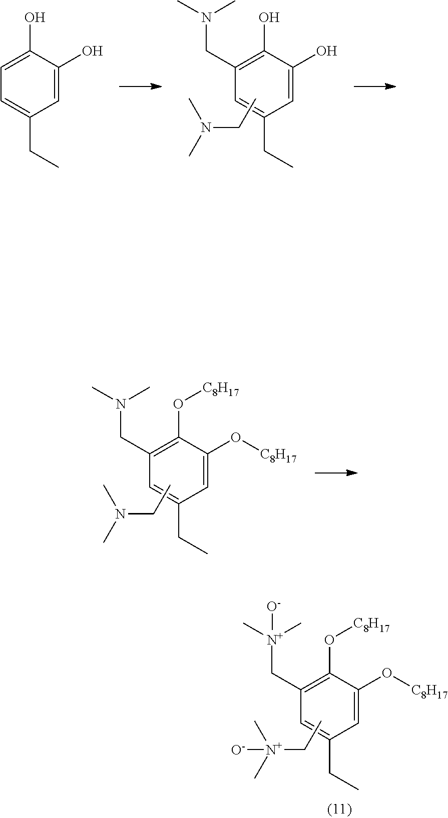

### Step 1:

An aqueous solution of 4-ethylcatechol (1.0 g, 7.24 mmol) was added dropwise and under argon atmosphere to a 40 wt % aqueous solution of dimethylamine (0.98 g, 21.7 mmol) in an iced water bath within 15 minutes with constant stirring. 10 ml of a 37 wt % aqueous solution of paraformaldehyde (0.65 g, 21.7 mmol) were added in 5 aliquots every 10 min and stirred for 2 h in an iced water bath and then for 58 h at room temperature. The reaction mixture was then extracted 5 times with 10 ml of Et₂O, and the combined organic phases were washed 5 times with 5 ml of water and then concentrated in vacuo on a rotary evaporator, whereafter the residue was completely dried in a vacuum desiccator, giving a mixture of the twice aminomethylated intermediates, 3,4- and 3,6-bis(dimethylaminomethyl)-5-ethylcatechol as an off-white powder (yield: 1.20 g; 65.5% of theory).

### Step 2:

The reaction was carried out in an analogous manner to that in Example 1, Variant 2.2, except that it was carried out under argon atmosphere for 12 h and that flash chromatography was conducted using a petroleum ether/ethyl acetate gradient, giving a mixture of 1,1′-(2,3-dioctyloxy-5-ethyl-1,4-phenylene)-bis(N,N-dimethylmethanamine) and 1,1′-(2,3-dioctyloxy-5-ethyl-1,6-phenylene)-bis(N,N-dimethylmethanamine) as a yellow oil (yield: 0.15 g; 12.3% of theory).

¹H NMR: δ_{H} (300 MHz, Chloroform-d) δ 6.91 (s, 1H), 3.92 (dt, J=8.2, 6.7 Hz, 4H), 3.40 (d, J=7.1 Hz, 4H), 2.71 (q, J=7.5 Hz, 2H), 2.24 (d, J=6.6 Hz, 12H), 1.75 (dt, J=8.3, 6.3 Hz, 4H), 1.53-1.39 (m, 4H), 1.36-1.25 (m, 18H), 1.19 (t, J=7.5 Hz, 3H), 0.94-0.83 (m, 6H). ¹³C NMR: δ_{C} (75 MHz, Chloroform-d) 151.5, 148.9, 139.9, 131.4, 129.7, 125.3, 73.4 (d, J=2.2 Hz), 58.2, 54.2, 45.7, 32.0, 30.7 (d, J=2.0 Hz), 29.7, 29.5, 26.4 (d, J=1.5 Hz), 25.3, 22.8, 15.7, 14.3. Elemental Analysis: Expected: C, 75.57; H, 11.84; N, 5.88; Found: C, 75.34; H, 11.85; N, 5.56; HRMS: (ESI⁺, m/z) calculated for C₃₀H₅₇N₂O₂ [M+H]⁺: 477.4420; found: 477.441284.

### Step 3:

The reaction was carried out in an analogous manner to that in Example 1, except that 0.25 mmol of the amine mixture were used; however, as a result of side reactions, the title compounds (11) were obtained in a mixture with several by-products. The optimization of this oxidation reaction is currently the subject of the inventors' research.

### Example 12

### Preparation of 1,1′-(5-ethyl-2-octyloxy-1,3-phenylene)-bis(N,N-dimethylmethanamine N-oxide) (12)

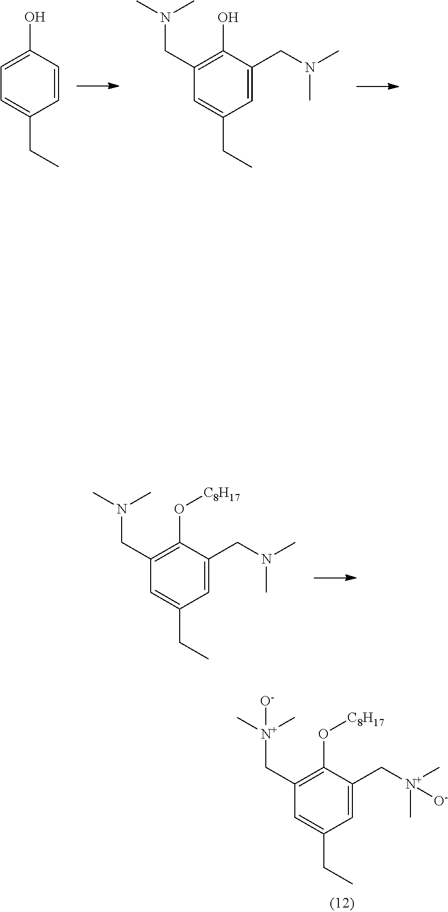

### Step 1:

An aqueous solution of 4-ethylphenol (6.11 g, 50 mmol) was added dropwise to a 40 wt % aqueous solution of dimethylamine (6.76 g, 150 mmol) in an iced water bath within 15 minutes with constant stirring. Paraformaldehyde (4.50 g, 150 mmol) was added in aliquots of 1.5 g every 10 min and stirred for 2 h in an iced water bath and then for 58 h at room temperature. The reaction mixture was then extracted 5 times with 25 ml of petroleum ether, and the combined organic phases were washed 5 times with 4 ml of water and then concentrated in vacuo on a rotary evaporator, whereafter the residue was completely dried in a vacuum desiccator. The twice aminomethylated intermediate, 2,6-bis(dimethylaminomethyl)-4-ethylphenol, was obtained as a viscous, clear oil (yield: 11.02 g; 93.3% of theory).

### Step 2:

The reaction was carried out in an analogous manner to that in Example 1, Variant 2.2, except that the KOH was added in two portions (0.28 g each at the start and after 2 h) and that flash chromatography was conducted using a petroleum ether/ethyl acetate gradient, giving 1,1′-(5-ethyl-2-octyloxy-1,3-phenylene)-bis(N,N-dimethylmethanamine) as a yellow oil (yield: 1.54 g; 8.6% of theory).

### Step 3:

The reaction was carried out in an analogous manner to that in Example 1, giving the title compound (12) as a viscous, clear, yellow oil (yield: 1.12 g; 97.9% of theory).

¹H NMR: δ_{H} (300 MHz, Chloroform-d) 7.51 (s, 2H, C2, C4), 4.48 (s, 4H, C8, C11), 3.71 (t, J=6.8, 6.8 Hz, 2H, C19), 3.16 (s, 12H, C13, C14, C16, C17), 2.67-2.54 (m, 2H, C9), 1.89-1.76 (m, 2H, C20), 1.45-1.34 (m, 2H, C21), 1.33-1.04 (m, 14H, C10, C22-C25), 0.85 (t, J=6.7 Hz, 3H, C26). ¹³C NMR: δ_{C} (75 MHz, Chloroform-d) 156.4 (C6), 141.1 (C3), 136.7 (C1, C5), 124.0 (C2, C4), 69.4 (C8, C11), 57.6 (C13, C14, C16, C17), 31.9 (C20), 30.4 (C21), 29.5 (C22), 29.3 (C23), 28.0 (C9), 26.2 (C24), 22.7 (C25), 15.3 (C10), 14.2 (C26). HRMS: (ESI⁺, m/z) calculated for C₂₂H₄₁NO₃ [M+H]⁺: 381.31172; found: 381.310922.

### Example 13

### Preparation of 1,1′-(2-dodecyloxy-5-ethyl-1,3-phenylene)-bis(N,N-dimethylmethanamine N-oxide) (13)

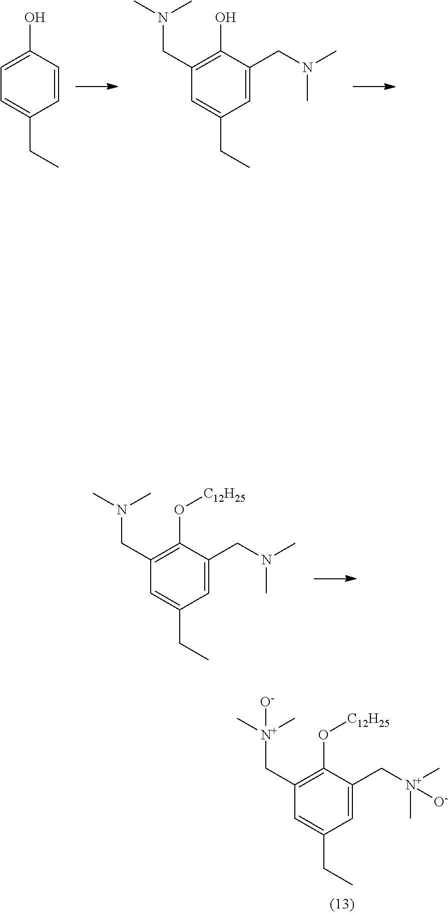

### Step 1:

The synthesis and the product were identical to Example 12.

### Step 2:

The reaction was carried out in an analogous manner to that in Example 1, Variant 2.2, except that 1-bromododecane was used instead of 1-bromooctane, that the KOH was added in two portions (0.28 g each at the start and after 2 h) and that flash chromatography was conducted using a petroleum ether/ethyl acetate gradient, giving 1,1′-(2-dodecyloxy-5-ethyl-1,3-phenylene)-bis(N,N-dimethylmethanamine) as a yellow oil (yield: 1.53 g; 75.7% of theory).

### Step 3:

The reaction was carried out in an analogous manner to that in Example 1, except that only 2.5 mmol of 1,1′-(2-dodecyloxy-5-ethyl-1,3-phenylene)-bis(N,N-dimethylmethanamine) were used, giving the title compound (13) as an off-white, waxy solid (yield: 0.70 g; 96.1% of theory).

¹H NMR: δ_{H} (300 MHz, Chloroform-d) 7.53 (s, 2H, C2, C4), 4.49 (s, 4H, C8, C11), 3.74 (t, J=6.8 Hz, 2H, C19), 3.19 (s, 12H, C13, C14, C16, C17), 2.64 (q, J=7.6 Hz, 2H, C9), 1.84 (t, J=7.4 Hz, 2H, C20), 1.46-1.37 (m, 2H, C21), 1.32-1.17 (m, 22H, C10, C22-C29), 0.87 (t, J=6.9 Hz, 3H, C30). ¹³C NMR: δ_{C} (75 MHz, Chloroform-d) 156.4 (C6), 141.2 (C3), 136.8 (C1, C5), 123.9 (C2, C4), 69.4 (C8, C11), 57.6 (C13, C14, C16, C17), 32.0 (C20), 30.5 (C21), 29.8 (C22, C24, C25), 29.6 (C26), 29.5 (C27), 28.1 (C9), 26.3 (C28), 22.8 (C29), 15.4 (C10), 14.3 (C30). HRMS: (ESI⁺, m/z) calculated for C₂₆H₄₉NO₃ [M+H]⁺: 437.37432; found: 437.373256.

### Example 14

### Preparation of 1,1′-(5-ethyl-2-hexadecyloxy-1,3-phenylene)-bis(N,N-dimethylmethanamine N-oxide) (14)

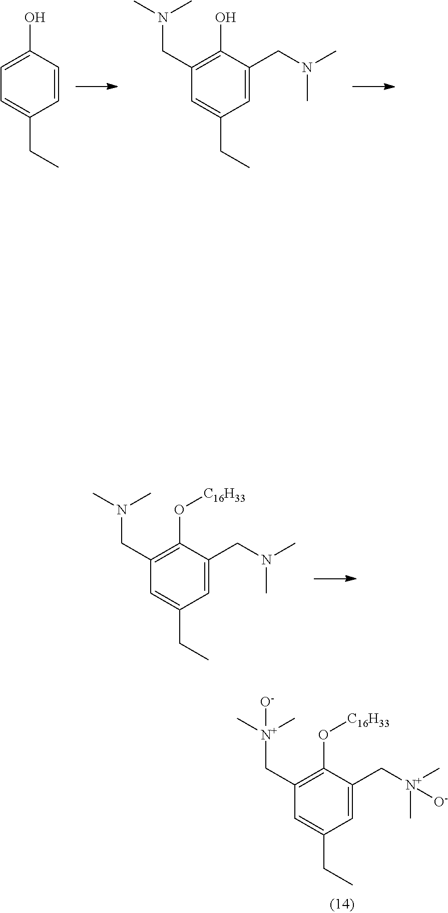

### Step 1:

The synthesis and the product were identical to Example 12.

### Step 2:

The reaction was carried out in an analogous manner to that in Example 13, except that 1-bromohexadecane was used instead of 1-bromooctane, giving 1,1′-(5-ethyl-2-hexadecyloxy-1,3-phenylene)-bis(N,N-dimethylmethanamine) as a yellow oil (yield: 1.57 g; 68.1% of theory).

### Step 3:

The reaction was carried out in an analogous manner to that in Example 13, giving the title compound (14) as an off-white, waxy solid (yield: 1.20 g; 97.2% of theory).

¹H NMR: δ_{H} (300 MHz, Chloroform-d) 7.54 (s, 2H, C2, C4), 4.50 (s, 4H, C8, C11), 3.74 (t, J=6.8 Hz, 2H, C19), 3.17 (s, 12H, C13, C14, C16, C17), 2.65 (q, J=7.5 Hz, 2H, C9), 1.88-1.82 (m, 2H, C20), 1.48-1.39 (m, 2H, C21), 1.30-1.21 (m, 29H, C10, C22-C33), 0.86 (t, J=7.0 Hz, 3H, C34). ¹³C NMR: δ_{C} (75 MHz, Chloroform-d) 156.4 (C6), 141.2 (C3), 136.7 (C1, C5), 124.2 (C2, C4), 69.6 (C8, C11), 57.8 (C13, C14, C16, C17), 32.0 (C20), 30.5 (C21), 30.0-29.3 (m) (C22-C31), 28.1 (C9), 26.3 (C32), 22.8 (C33), 15.4 (C10), 14.3 (C34). HRMS: (ESI⁺, m/z) calculated for C₃₀H₅₇N₂O₃ [M+H]⁺: 493.43692; found: 493.436311.

### Example 15

### Preparation of 1,1′-(5-ethyl-2-octadecyloxy-1,3-phenylene)-bis(N,N-dimethylmethanamine N-oxide) (15)

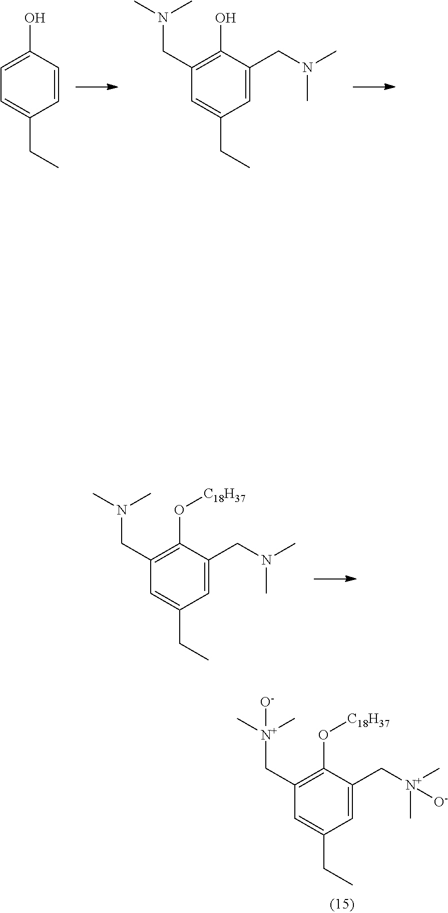

### Step 1:

The synthesis and the product were identical to Example 12.

### Step 2:

The reaction was carried out in an analogous manner to that in Example 13, except that 1-bromooctadecane was used instead of 1-bromododecane, giving 1,1′-(5-ethyl-2-octadecyloxy-1,3-phenylene)-bis(N,N-dimethylmethanamine) as an off-white, waxy solid (yield: 1.61 g; 66.0% of theory).

### Step 3:

The reaction was carried out in an analogous manner to that in Example 13, giving the title compound (15) as a yellowish, waxy solid (yield: 1.00 g; 96.2% of theory).

¹H NMR: δ_{H} (300 MHz, Chloroform-d) 7.51 (s, 2H, C2, C4), 4.40 (s, 4H, C8, C11), 3.72 (t, J=6.8 Hz, 2H, C19), 3.08 (s, 12H, C13, C14, C16, C17), 2.65 (q, J=7.7 Hz, 2H, C9), 1.88-1.77 (m, 2H, C20), 1.45-1.36 (m, 2H, C21), 1.26-1.18 (m, 33H, C10, C22-C35), 0.84 (t, J=7.0 Hz, 3H, C36). ¹³C NMR: δ_{C} (75 MHz, Chloroform-d) 156.2 (C6), 141.2 (C3), 136.3 (C1, C5), 124.6 (C2, C4), 70.2 (C8, C11), 58.1 (C13, C14, C16, C17), 32.0 (C20), 30.5 (C21), 29.9-29.6 (m) (C₂₂-C₃₁), 29.5 (C32), 29.4 (C33), 28.1 (C9), 26.2 (C34), 22.8 (C35), 15.3 (C10), 14.2 (C36); HRMS: (ESI⁺, m/z) calculated for C₃₂H₆₁NO₃ [M+H]⁺: 521.46822; found: 521.467246.

### Example 16

### Preparation of 1,4-bis(5-ethyl-3-methoxy-2-octyloxybenzyl)piperazine-1,4-dioxide (16)

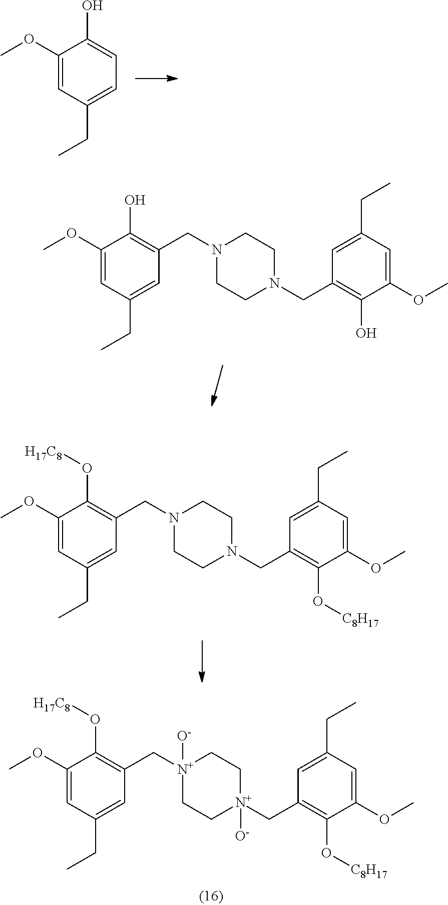

### Step 1:

An aqueous solution of 4-ethylguaiacol (3.04 g, 20 mmol) was added dropwise to an aqueous solution (10 ml) of piperazine (1.29 g, 15 mmol) in an iced water bath within 15 minutes with constant stirring. A 37 wt % aqueous solution of formaldehyde (0.90 g, 30 mmol) was added in aliquots of 0.1 g every 10 min and stirred for 3 h in an iced water bath and then for 9 h at room temperature. The precipitated solid was then filtered off (glass frit, porosity 4), suspended in 10 ml petroleum ether, mixed by sonication, then centrifuged off and completely dried in a vacuum desiccator, giving the dimeric intermediate, 6,6′-(piperazine-1,4-dimethylene)-bis(4-ethyl-2-methoxyphenol), in the form of white needles (yield: 3.40 g; 82.1% of theory).

### Step 2:

The reaction was carried out in an analogous manner to that in Example 1, Variant 2.2, except that 2.12 g (11 mmol) 1-bromooctane were used, that 40 ml 2-MeTHF were used as a solvent, and that flash chromatography was conducted using a petroleum ether/ethyl acetate gradient, giving 1,4-bis(5-ethyl-3-methoxy-2-octyloxybenzyl)piperazine as a white powder (yield: 1.45 g; 45.4% of theory).

### Step 3:

The reaction was carried out in an analogous manner to that in Example 1, except that only 1 mmol of 1,4-bis(5-ethyl-3-methoxy-2-octyloxybenzyl)piperazine was used and that methyl formate (15 ml) was added as an additional solvent, giving the title compound (16) as a white, waxy solid (yield: 0.21 g; 31.6% of theory).

¹H NMR: δ_{H} (300 MHz, Chloroform-d) 6.94 (d, J=2.0 Hz, 2H), 6.77 (d, J=2.0 Hz, 2H), 4.45 (s, 4H), 4.28 (d, J=8.3 Hz, 4H), 3.93 (t, J=7.0 Hz, 4H), 3.82 (s, 6H), 3.02 (d, J=8.1 Hz, 4H), 2.59 (q, J=7.6 Hz, 4H), 1.74 (q, J=7.0 Hz, 4H), 1.44-1.15 (m, 26H), 0.93-0.82 (m, 6H). ¹³C NMR: δ_{C} (75 MHz, Chloroform-d) 152.4, 139.8, 125.2, 122.4, 114.1, 73.7, 58.6, 55.9, 32.0, 30.4, 29.5, 29.4, 28.6, 26.0, 22.8, 15.4, 14.3. HRMS: (ESI⁺, m/z) calculated for C₄₀H₆₇N₂O₆ [M+H]⁺: 671.49991; found: 671.498929.

### Example 17

### Preparation of 1,4-bis(2-dodecyloxy-5-ethyl-3-methoxybenzyl)piperazine-1,4-dioxide (17)

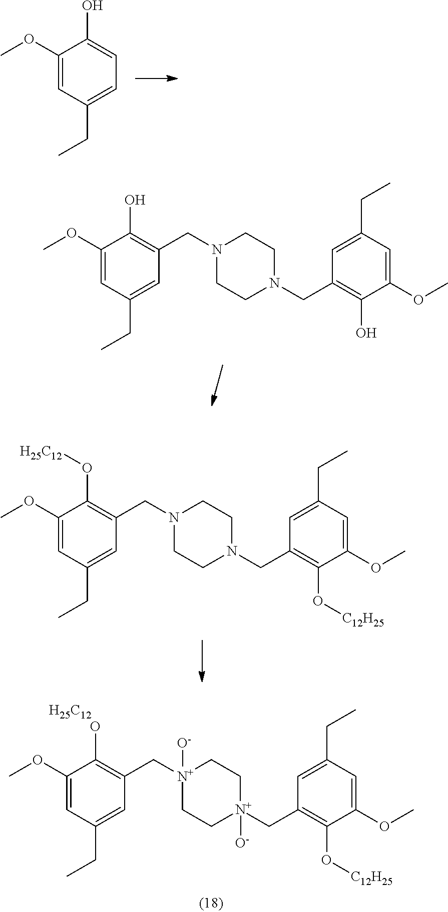

### Step 1:

The synthesis and the product were identical to Example 16.

### Step 2:

The reaction was carried out in an analogous manner to that in Example 16, except that 1-bromododecane was used instead of 1-bromooctane, giving 1,4-bis(2-dodecyloxy-5-ethyl-3-methoxybenzyl)piperazine as a white powder (yield: 1.61 g; 79.6% of theory).

¹H NMR: δ_{H} (600 MHz, Chloroform-d) 6.77 (2H, d, J 2.0), 6.63 (2H, d, J 2.0), 3.89 (4H, t, J 6.8), 3.82 (6H, s), 3.52 (4H, s), 2.66-2.32 (12H, m), 1.75 (4H, p, J 6.9), 1.48-1.40 (4H, m), 1.37-1.23 (36H, m), 1.22 (6H, t, J 7.6), 0.88 (6H, t, J 7.0). ¹³C NMR: δ_{C} (151 MHz, Chloroform-d) 152.7, 145.2, 139.5, 131.8, 121.9, 110.9, 73.6, 56.8, 55.9, 53.4, 32.1, 30.5, 29.9-29.8 (m), 29.7, 29.5, 28.9, 26.3, 22.8, 15.8, 14.3. Elemental Analysis: Expected: C, 76.75; H, 11.00; N, 3.73; Found: C, 76.18; H, 10.51; N, 3.67; HRMS: (ESI⁺, m/z) calculated for C₄₈H₈₃N₂O₄ [M+H]⁺: 751.63474; found: 751.63595.

### Step 3:

The reaction was carried out in an analogous manner to that in Example 1; however, as a result of the poor solubility of 1,4-bis(2-dodecyloxy-5-ethyl-3-methoxybenzyl)piperazine in water, only very low conversion was observed. The optimization of this oxidation reaction using methyl formate as an additional solvent is currently the subject of the inventors' research.

### Example 18

### Preparation of N,N′-bis(5-ethyl-3-methoxy-2-octyloxybenzyl)-N,N′-dimethylethane-1,2-diamine di-N-oxide (18)

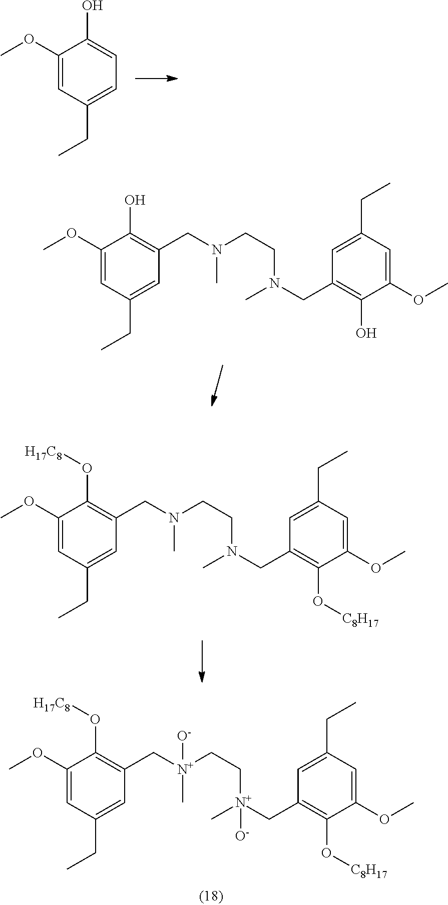

### Step 1:

The reaction was carried out in an analogous manner to that in Example 16, except that N,N′-dimethylethylenediamine was used instead of piperazine, giving 6,6′-(N,N′-dimethylethan-1,2-diamine-N,N′-dimethylene)-bis(4-ethyl-2-methoxyphenol) as a yellowish oil (yield: 1.90 g; 91.4% of theory).

### Step 2:

The reaction was carried out in an analogous manner to that in Example 16, giving N,N′-bis(5-ethyl-3-methoxy-2-octyloxybenzyl)-N,N′-dimethylethan-1,2-diamine as an off-white powder (yield: 0.91 g; 28.5% of theory).

### Step 3:

The reaction was carried out in an analogous manner to that in Example 16 using methyl formate (15 ml) as an additional solvent, giving the title compound (17) as a white, waxy solid. However, since decomposition reactions of the diamine occurred in the course of the oxidation, the desired product was obtained in admixture with a number of by-products which could hardly be separated even using column chromatography. The optimization of this oxidation reaction is currently the subject of the inventors' research.

### Example 19

### Tests Regarding the Suitability of the Isolated Amine N-Oxide Compounds According to Formulae (I) and (II) as Surfactants

As a parameter for the surfactant properties of the novel amine-N-oxide compounds, as usual, the critical micelle concentration (CMC), i.e. the concentration of the protonated or cationic form of the surfactants at which micelles can form was used and was determined using a K100C Force Tensiometer from Krüss Scientific according to the Wilhelmy plate method at 25° C. and pH 3. For comparison, dodecyldimethylamine N-oxide (“C1”) was measured under the same conditions. The results are given in Table 1 below, with lower values indicating a stronger surfactant effect of the respective substance.

TABLE 1

| Compound | CMC (mmol/l) |
|---|---|
| (1) | 4.1 × 10³ |
| (2) | 5.7 × 10² |
| (3)/(3)* | 89/19 |
| (4) | 5.2 |
| (5) | 4.0 |
| (6) | 1.3 |
| (7) | 36 |
| (8) | 1.3 × 10² |
| (13) | 1.9 × 10² |
| (14) | 75 |
| (15) | 29 |
| C1 | 4.2 × 10³ |

It can be seen that the examples according to the invention, with the exception of Example 1, have lower—and in the majority significantly lower—CMC values than the comparison substance C1, which is used in a large number of commercially available products. However, the highest CMC value of 4.1 mol/l for the amine N-oxide (1) from Example 1, which contains the lowest number of carbon atoms in the radicals R¹ to R⁵, also proves its suitability as a surfactant, since this value is almost identical to that of the commercially available compound C1.

Due to the analogies or high similarities in the substitution patterns of the other compounds according to the invention which have not yet been tested, the relevant person skilled in the art can expect that a strong surfactant effect will also be detectable for the majority of these.

In addition, when solutions of these amine N-oxide surfactants were examined in double distilled water at concentrations of 10 mg/ml each using cryogenic electron microscopy, a previously unknown phenomenon was surprisingly discovered: The ends of the worm-like micelles formed by the surfactants are folded, i.e. they are not present in an elongated form, as has been consistently reported so far. As an example, a cryogenic electron microscope image of such a solution of the amine N-oxide compound (13) from Example 13 is shown in FIG. 1 together with a schematic representation of the worm-like micelles observed therein, which have a size of 40 nm to 76 nm.

The present invention thus provides a method for the preparation of novel amine N-oxide compounds which comprises only three relatively simple synthesis steps and through which the novel amine N-oxides can be obtained in very good yields and in an economical and environmentally friendly manner, the vast majority of which are suitable for use as surfactants.

1. A method for preparing an amine N-oxide compound according to the following formula (I) or (II): 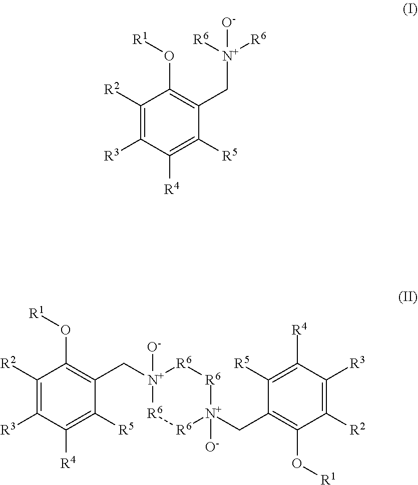
wherein
each R¹ is selected from linear, branched or cyclic hydrocarbon radicals having 4 to 26 carbon atoms, wherein optionally at least one carbon atom may be replaced by an oxygen or sulfur atom;
R², R³ and R⁵ are each independently selected from hydrogen, R¹—O—, R⁸, and in formula (I) also from —CH₂—N⁺(O⁻)R⁶R⁶, wherein R¹ represents a linear, branched or cyclic hydrocarbon radical having 1 to 26 carbon atoms, wherein optionally at least one carbon atom may be replaced by an oxygen or sulfur atom;
R⁴ is selected from hydrogen and R⁸; and
each radical R⁶ is independently selected from saturated, linear or branched hydrocarbon radicals having 1 to 6 carbon atoms, wherein optionally at least one carbon atom may be replaced by a nitrogen, oxygen or sulfur atom;
wherein optionally two radicals R⁶ being bound to the same nitrogen atom may be connected to form a five- or six-membered, nitrogen-containing ring, or
wherein optionally one or both radical(s) R⁶ of an amine N-oxide moiety —N⁺(O⁻)R⁶R⁶ may be linked to one or both radical(s) R⁶ of such a moiety of another molecule of formula (I), thus forming a bridge having the structure 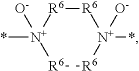
wherein the dashed line designates an optional bond between the two radicals R and the asterisks designate the connections of the bridge to the two aromatic rings, thereby forming a dimer according to formula (II);
wherein the method comprising the following steps:
1) reacting a phenol derivative according to the following formula (III): 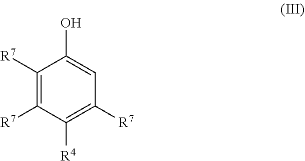
wherein each R⁷ is independently selected from hydrogen, hydroxy, and R⁸,
with a secondary amine HNR⁶R⁶ by means of an amino alkylation reaction according to Betti/Mannich in the presence of formaldehyde in a polar solvent, thereby substituting the hydrogen atom in the ortho position to the phenolic OH group, and optionally another substitutable hydrogen atom R⁷ of the phenol derivative of formula (II), by a —CH₂—NR⁶R⁶ moiety (each), resulting in a corresponding Betti base according to formula (IV) or (V): 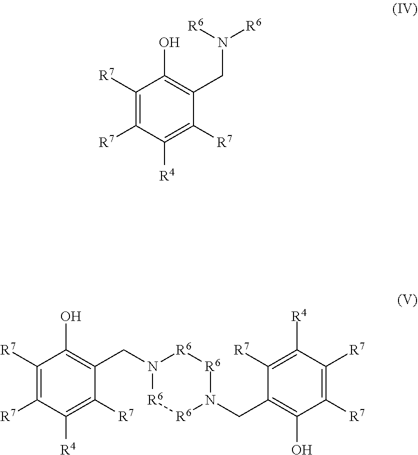
wherein each R⁷ is independently selected from hydrogen, hydroxy, R⁸, and in formula (IV) also from —CH₂—N⁶R⁶;
2) reacting the (two) phenolic OH group(s), and optionally any further free OH group(s) R⁷, of the respective Betti base of formula (IV) or (V) with a compound according to the formula R¹—X, wherein X represents a leaving group selected from halides and sulfonates, by means of an etherification reaction according to Williamson in the presence of a base, in an organic solvent or without any solvent, resulting in a corresponding ether according to formula (VI) or (VII): 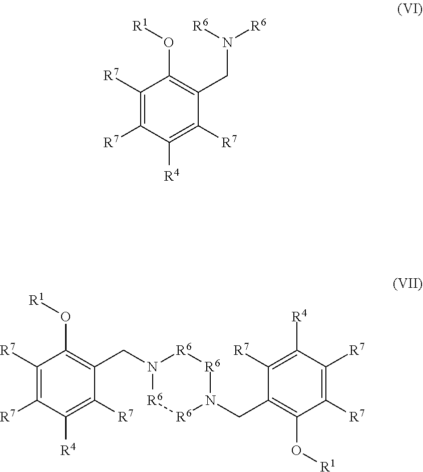
wherein each R⁷ is independently selected from hydrogen, R¹—O—, R⁸, and in formula (VI) also from —CH₂—NR⁶R⁶; and
3) oxidizing any amino groups —NR⁶R⁶ of the respective ether of formula (VI) or (VII) by reaction with an oxidizing agent in water, an organic solvent or a mixture thereof, resulting in the amine N-oxide compound of formula (I) or (II).

2. The method according to claim 1, wherein, in step 1), the phenol derivative of formula (III) is reacted with 1.5 equivalents each of the secondary amine and of formaldehyde,
the reaction being optionally carried out in water at room temperature.

3. The method according to claim 1, wherein, in step 2),
a solid-liquid phase-transfer reaction is carried out using a solid base and in the presence of a phase transfer catalyst; and/or
chloride or bromide is used as said leaving group X; and/or
an anhydrous solvent is used.

4. The method according to claim 3, wherein, in step 2),
powdered KOH is used a said solid base;
tetra-n-butylammonium bromide (TBAB) is used as said phase-transfer catalyst;
bromide is used as said leaving group X; and
anhydrous 2-methyltetrahydrofuran is used as a solvent.

5. The method according to claim 1, wherein, in step 3),
an aqueous solution of H₂O₂ is used as said oxidation agent, wherein optionally formic acid methyl ester may be added as an additional solvent; and/or
the ether of formula (VI) or (VII) is reacted with 2.5 to 3 equivalents of H₂O₂.

6. An amine N-oxide compound according to the following formula (I) or (II), prepared according to the method according to claim 1: 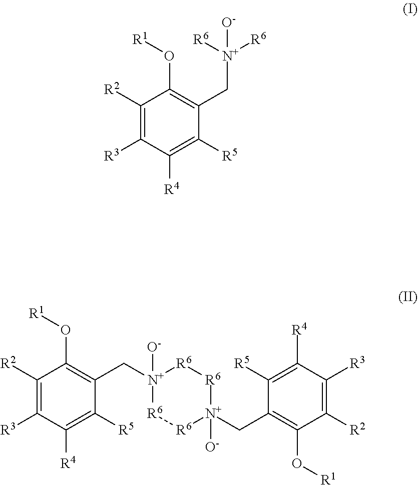
wherein
each R¹ is selected from linear, branched or cyclic hydrocarbon radicals having 4 to 26 carbon atoms, wherein optionally at least one carbon atom may be replaced by an oxygen or sulfur atom;
R², R³ and R⁵ are each independently selected from hydrogen, R¹—O—, R⁸, and in formula (I) also from —CH₂—N⁺(O⁻)R⁶R⁶, wherein R⁸ represents a linear, branched or cyclic hydrocarbon radical having 1 to 26 carbon atoms, wherein optionally at least one carbon atom may be replaced by an oxygen or sulfur atom;
R⁴ is selected from hydrogen and R⁸; and
each radical R⁶ is independently selected from saturated, linear or branched hydrocarbon radicals having 1 to 6 carbon atoms, wherein optionally at least one carbon atom may be replaced by a nitrogen, oxygen or sulfur atom;
wherein optionally two radicals R⁶ being bound to the same nitrogen atom may be connected to form a five- or six-membered, nitrogen-containing ring, or
wherein optionally one or both radical(s) R⁶ of an amine N-oxide moiety —N⁺(O⁻)R⁶R⁶ may be linked to one or both radical(s) R⁶ of such a moiety of another molecule of formula (I), thus forming a bridge having the structure 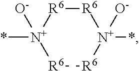
wherein the dashed line designates an optional bond between the two radicals R⁶ and the asterisks designate the connections of the bridge to the two aromatic rings, thereby forming a dimer according to formula (II).

7. The amine N-oxide compound according to claim 6, wherein
R¹ is C₆-C₂₂ alkyl; and/or
R² is selected from C₁-C₂₂ alkyl, C₁-C₂₂ alkoxy, and —CH₂—N⁺(O⁻)R⁶R⁶; and/or
R³ and R⁵ are each selected from hydrogen and —CH₂—N⁺(O⁻)R⁶R⁶; and/or
R⁴ is selected from hydrogen, C₁-C₄ alkyl, and C₁-C₄ alkoxy.

8. The amine N-oxide compound according to claim 7, wherein
R¹ is C₈-C₁₈ alkyl; and/or
R² is selected from C₁-C₁₈ alkoxy and —CH₂—N⁺(O⁻)R⁶R⁶; and/or
R⁴ and R⁵ are each selected from hydrogen and C₁-C₄ alkyl.

9. The amine N-oxide compound according to claim 7, wherein
R¹ is C₈-C₁₈ alkyl;
R² is C₁-C₁₈ alkoxy;
one of R³ and R⁵ is hydrogen and the other is —CH₂—N⁺(O⁻)R⁶R⁶; and
R⁴ is C₁-C₄ alkyl.

10. The amine N-oxide compound according to claim 7, wherein
R¹ is C₈-C₁₈ alkyl;
R² is C₁-C₁₈ alkoxy;
R³ and R⁵ each are hydrogen; and
R⁴ is C₁-C₄ alkyl.

11. The amine N-oxide compound according to claim 10, wherein
R² is methoxy; and
R⁴ is ethyl or propyl.

12. The amine N-oxide compound according to claim 7, wherein
R¹ is C₈-C₁₈ alkyl;
R² is —CH₂—N⁺(O⁻)R⁶R⁶;
R³ and R⁵ each are hydrogen; and
R⁴ is C₁-C₄ alkyl.

13. The amine N-oxide compound according to claim 6, wherein
each radical R⁶ is independently selected from methyl, ethyl, and dimethylaminoethyl; and/or
two radicals R⁶ being bound to the same nitrogen atom are connected to form, together with the nitrogen atom, one of the following groups: 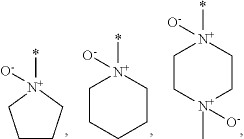
wherein the asterisks designate the respective connections to the aromatic ring; and/or
all radicals R⁶ are methyl and one or both methyl group(s) of a moiety —N⁺(O⁻)(CH₃)₂ is/are linked to one or both methyl group(s) R⁶ of such a moiety of another molecule of formula (I), thus forming a bridge having the structure 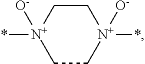
wherein the dashed line designates an optional bond between the two methyl groups and the asterisks designate the connections of the bridge to the two aromatic rings, thereby forming a dimer of the amine N-oxide compound according to formula (II).

14. The amine N-oxide compound according to claim 6, wherein it is selected from the following compounds:
N,N-dimethyl-1-(5-ethyl-3-methoxy-2-octyloxyphenyl)methanamine N-oxide (1) 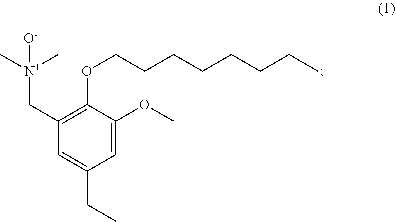 [SMILES: CCCCCCCCOc1c(C[N+](C)(C)[O-])cc(CC)cc1OC]
N,N-dimethyl-1-(2-decyloxy-5-ethyl-3-methoxyphenyl)methanamine N-oxide (2) 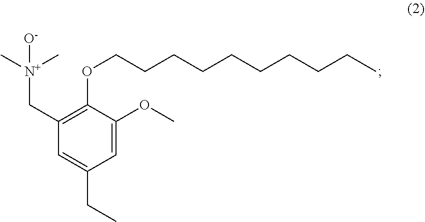 [SMILES: CCCCCCCCCCOc1c(C[N+](C)(C)[O-])cc(CC)cc1OC]
N,N-dimethyl-1-(2-dodecyloxy-5-ethyl-3-methoxyphenyl)methanamine N-oxide (3) 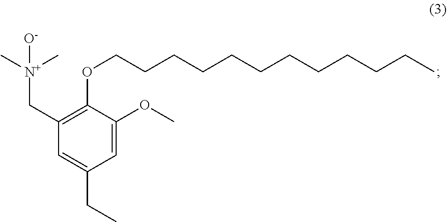 [SMILES: CCCCCCCCCCCCOc1c(C[N+](C)(C)[O-])cc(CC)cc1OC]
N,N-dimethyl-1-(5-ethyl-3-methoxy-2-tetradecyloxyphenyl)methanamine N-oxide (4) 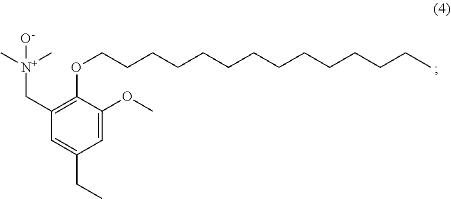 [SMILES: CCCCCCCCCCCCCCOc1c(C[N+](C)(C)[O-])cc(CC)cc1OC]
N,N-dimethyl-1-(5-ethyl-2-hexadecyloxy-3-methoxyphenyl)methanamine N-oxide (5) 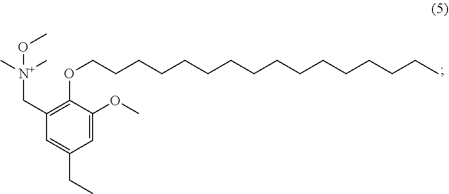 [SMILES: CCCCCCCCCCCCCCCCOc1c(C[N+](C)(C)OC)cc(CC)cc1OC]
N,N-dimethyl-1-(5-ethyl-3-methoxy-2-octadecyloxyphenyl)methanamine N-oxide (6) 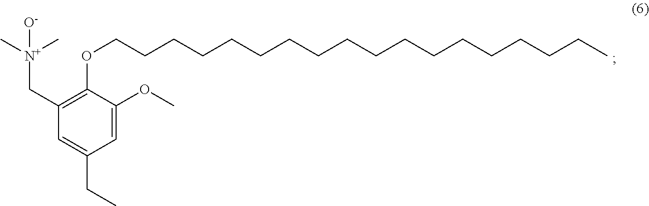 [SMILES: CCCCCCCCCCCCCCCCCCOc1c(C[N+](C)(C)[O-])cc(CC)cc1OC]
1-(2-dodecyloxy-5-ethyl-3-methoxybenzyl)pyrrolidine-1-oxide (7) 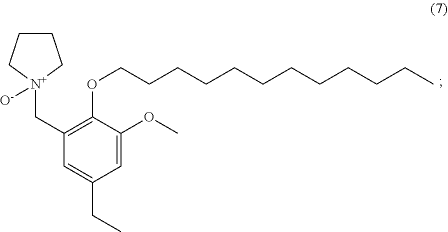 [SMILES: CCCCCCCCCCCCOc1c(C[N+]2([O-])CCCC2)cc(CC)cc1OC]
1-(2-dodecyloxy-5-ethyl-3-methoxybenzyl)piperidine-1-oxide (8) 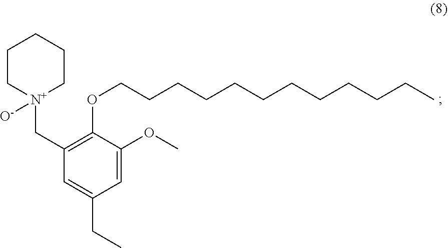 [SMILES: CCCCCCCCCCCCOc1c(C[N+]2([O-])CCCCC2)cc(CC)cc1OC]
1-(2-dodecyloxy-5-ethyl-3-methoxybenzyl)-4-methylpiperazine-1,4-dioxide (9) 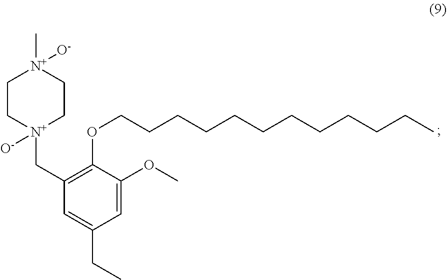 [SMILES: CCCCCCCCCCCCOc1c(C[N+]2([O-])CC[N+](C)([O-])CC2)cc(CC)cc1OC]
N,N-dimethyl-N′-(2-dodecyloxy-5-ethyl-3-methoxybenzyl)-N′-methylethane-1,2-diamine di-N-oxide (10) 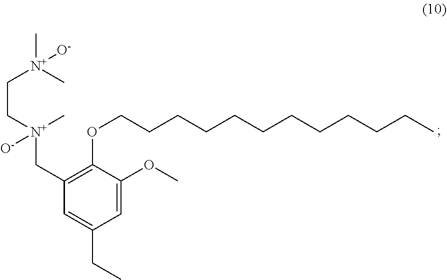 [SMILES: CCCCCCCCCCCCOc1c(C[N+](C)([O-])CC[N+](C)(C)[O-])cc(CC)cc1OC]
1,1′-(2,3-dioctyloxy-5-ethyl-1,4-phenylene)-bis(N,N-dimethylmethanamine N-oxide) and 1,1′-(2,3-dioctyloxy-5-ethyl-1,6-phenylene)-bis(N,N-dimethylmethanamine N-oxide) (11) 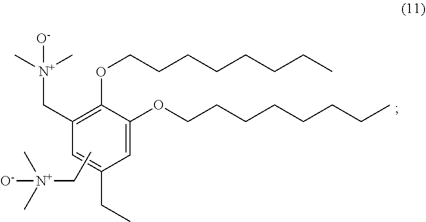
1,1′-(5-ethyl-2-octyloxy-1,3-phenylene)-bis(N,N-dimethylmethanamine N-oxide) (12) 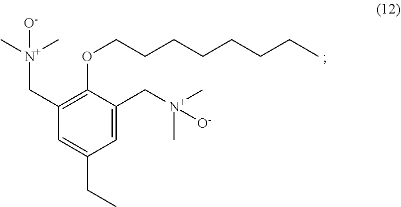 [SMILES: CCCCCCCCOc1c(C[N+](C)(C)[O-])cc(CC)cc1C[N+](C)(C)[O-]]
1,1′-(2-dodecyloxy-5-ethyl-1,3-phenylene)-bis(N,N-dimethylmethanamine N-oxide) (13) 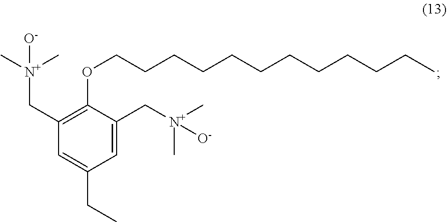 [SMILES: CCCCCCCCCCCCOc1c(C[N+](C)(C)[O-])cc(CC)cc1C[N+](C)(C)[O-]]
1,1′-(5-ethyl-2-hexadecyloxy-1,3-phenylene)-bis(N,N-dimethylmethanamine N-oxide) (14) 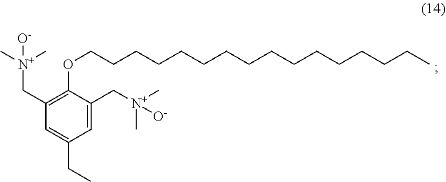 [SMILES: CCCCCCCCCCCCCCCCOc1c(C[N+](C)(C)[O-])cc(CC)cc1C[N+](C)(C)[O-]]
1,1′-(5-ethyl-2-octadecyloxy-1,3-phenylene)-bis(N,N-dimethylmethanamine N-oxide) (15) 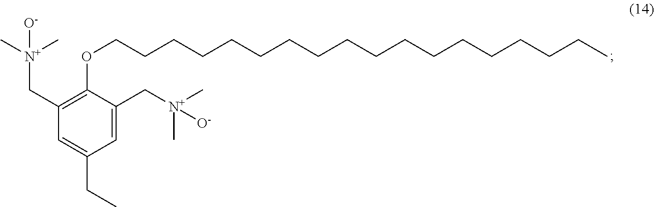 [SMILES: CCCCCCCCCCCCCCCCCCOc1c(C[N+](C)(C)[O-])cc(CC)cc1C[N+](C)(C)[O-]]
1,4-bis(5-ethyl-3-methoxy-2-octyloxybenzyl)piperazine-1,4-dioxide (16) 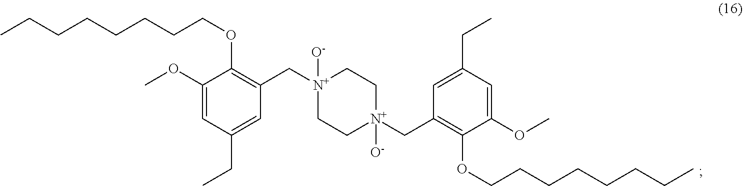 [SMILES: CCCCCCCCOc1c(C[N+]2([O-])CC[N+]([O-])(Cc3cc(CC)cc(OC)c3OCCCCCCCC)CC2)cc(CC)cc1OC]
1,4-bis(2-dodecyloxy-5-ethyl-3-methoxybenzyl)piperazine-1,4-dioxide (17) 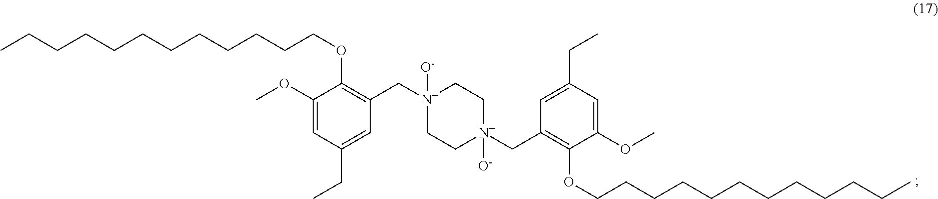 [SMILES: CCCCCCCCCCCCOc1c(C[N+]2([O-])CC[N+]([O-])(Cc3cc(CC)cc(OC)c3OCCCCCCCCCCCC)CC2)cc(CC)cc1OC]
N,N′-bis(5-ethyl-3-ethyl-2-octyloxybenzyl)-N,N′-dimethylethane-1,2-diamine di-N-oxide (18)

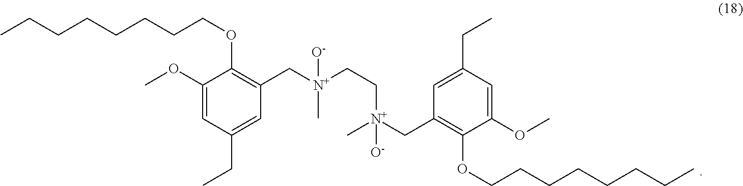 [SMILES: CCCCCCCCOc1c(C[N+](C)([O-])CC[N+](C)([O-])Cc2cc(CC)cc(OC)c2OCCCCCCCC)cc(CC)cc1OC]

15. A surfactant comprising the amine N-oxide compound of formula (I) or (II) according to claim 6, wherein the total number of carbon atoms of the radicals R¹ to R⁵ is at least 9.
